SEQUENCE LISTING (15 sequences)

SEQ ID NO: 1 - protein, 20 aa, Artificial
  Human HSP60 epitope
LVIIAEDVDGEALSTLVLNR

SEQ ID NO: 2 - protein, 20 aa, Artificial
  Human HSP60 epitope
GEVIVTKDDAMLLKGKGDKA

SEQ ID NO: 3 - protein, 20 aa, Artificial
  Human HSP60 epitope
KGDKAQIEKRIQEIIEQLDV

SEQ ID NO: 4 - protein, 20 aa, Artificial
  Human HSP60 epitope
NERLAKLSDGVAVLKVGGTS

SEQ ID NO: 5 - protein, 20 aa, Artificial
  Human HSP60 epitope
VGGTSDVEVNEKKDRVTDAL

SEQ ID NO: 6 - protein, 20 aa, Artificial
  Human HSP60 epitope
IVLGGGCALLRCIPALDSLT

SEQ ID NO: 7 - protein, 20 aa, Artificial
  Human HSP60 epitope
EIIKRTLKIPAMTIAKNAGV

SEQ ID NO: 8 - protein, 20 aa, Artificial
  Human HSP60 epitope
KNAGVEGSLIVEKIMQSSSE

SEQ ID NO: 9 - protein, 20 aa, Artificial
  Human HSP60 epitope
QSSSEVGYDAMAGDFVNMVE

SEQ ID NO: 10 - protein, 20 aa, Artificial
  human hsp60 epitope
KFGADARALMLQGVDLLADA

SEQ ID NO: 11 - protein, 15 aa, Mycobacterium tuberculosis
EESNTFGLQLELTEG

SEQ ID NO: 12 - DNA, 2691 bp, Homo sapiens
cgccatggagaccaacacccttcccaccgccactcccccttcctctcagggtccctgtcccctccagtgaatcccagaagactctggagagttctgagcagggggcggcactctggcctctgattggtccaaggaaggctggggggcaggacgggaggcgaaacccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagccgagccgacagagagcagggaaccgcatggccaaagccgcggcagtcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcgcctgatcggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtcctgatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtgggctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggccccaggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggccgacaagaagaaggtgctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtagattaggggcctttccaagattgctgtttttgttttggagcttcaagactttgcatttcctagtatttctgtttgtcagttctcaatttcctgtgtttgcaatgttgaaattttttggtgaagtactgaacttgcctttttttccggtttctacatgcagagatgaatttatactgccatcttacgactatttcttctttttaatacacttaactcaggccattttttaagttggttacttcaaagtaaataaactttaaaattcaagtgatgcccttttattcct

SEQ ID NO: 13 - DNA, 2912 bp, Homo sapiens
cagttgcttcagcgtcccggtgtggctgtgccgttggtcctgtgcggtcacttagccaagatgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaactttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgatctgtttgaaaacagaaagaaaaagaacaatatcaaattgtatgtacgcagagttttcatcatggataactgtgaggagctaatccctgaatatctgaacttcattagaggggtggtagactcggaggatctccctctaaacatatcccgtgagatgttgcaacaaagcaaaattttgaaagttatcaggaagaatttggtcaaaaaatgcttagaactctttactgaactggcggaagataaagagaactacaagaaattctatgagcagttctctaaaaacataaagcttggaatacacgaagactctcaaaatcggaagaagctttcagagctgttaaggtactacacatctgcctctggtgatgagatggtttctctcaaggactactgcaccagaatgaaggagaaccagaaacatatctattatatcacaggtgagaccaaggaccaggtagctaactcagcctttgtggaacgtcttcggaaacatggcttagaagtgatctatatgattgagcccattgatgagtactgtgtccaacagctgaaggaatttgaggggaagactttagtgtcagtcaccaaagaaggcctggaacttccagaggatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaagagacaactcaacaatgggttacatggcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtattgatgaagatgaccctactgctgatgataccagtgctgctgtaactgaagaaatgccaccccttgaaggagatgacgacacatcacgcatggaagaagtagactaatctctggctgagggatgacttacctgttcagtactctacaattcctctgataatatattttcaaggatgtttttctttatttttgttaatattaaaaagtctgtatggcatgacaactactttaaggggaagataagatttctgtctactaagtgatgctgtgataccttaggcactaaagcagagctagtaatgctttttgagtttcatgttggttctttcacagatggggtaacgtgcactgtaagacgtatgtaacatgatgttaactttgtgtggtctaaagtgtttagctgtcaagccggatgcctaagtagaccaaatcttgttattgaagtgttctgagctgtatcttgatgtttagaaaagtattcgttacatcttgtaggatctactttttgaacttttcattccctgtagttgacaattctgcatgtactagtcctctagaaataggttaaactgaagcaacttgatggaaggatctctccacagggcttgttttccaaagaaaagtattgtttggaggagcaaagttaaaagcctacctaagcatatcgtaaagctgttcaaatactcgagcccagtcttgtggatggaaatgtagtgctcgagtcacattctgcttaaagttgtaacaaatacagatgagt

SEQ ID NO: 14 - protein, 573 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 15 - DNA, 2246 bp, Homo sapiens
acgacctgtctcgccgagcgcacgccttgccgccgccccgcagaaatgcttcggttacccacagtctttcgccagatgagaccggtgtccagggtactggctcctcatctcactcgggcttatgccaaagatgtaaaatttggtgcagatgcccgagccttaatgcttcaaggtgtagaccttttagccgatgctgtggccgttacaatggggccaaagggaagaacagtgattattgagcagagttggggaagtcccaaagtaacaaaagatggtgtgactgttgcaaagtcaattgacttaaaagataaatacaagaacattggagctaaacttgttcaagatgttgccaataacacaaatgaagaagctggggatggcactaccactgctactgtactggcacgctctatagccaaggaaggcttcgagaagattagcaaaggtgctaatccagtggaaatcaggagaggtgtgatgttagctgttgatgctgtaattgctgaacttaaaaagcagtctaaacctgtgaccacccctgaagaaattgcacaggttgctacgatttctgcaaacggagacaaagaaattggcaatatcatctctgatgcaatgaaaaaagttggaagaaagggtgtcatcacagtaaaggatggaaaaacactgaatgatgaattagaaattattgaaggcatgaagtttgatcgaggctatatttctccatactttattaatacatcaaaaggtcagaaatgtgaattccaggatgcctatgttctgttgagtgaaaagaaaatttctagtatccagtccattgtacctgctcttgaaattgccaatgctcaccgtaagcctttggtcataatcgctgaagatgttgatggagaagctctaagtacactcgtcttgaataggctaaaggttggtcttcaggttgtggcagtcaaggctccagggtttggtgacaatagaaagaaccagcttaaagatatggctattgctactggtggtgcagtgtttggagaagagggattgaccctgaatcttgaagacgttcagcctcatgacttaggaaaagttggagaggtcattgtgaccaaagacgatgccatgctcttaaaaggaaaaggtgacaaggctcaaattgaaaaacgtattcaagaaatcattgagcagttagatgtcacaactagtgaatatgaaaaggaaaaactgaatgaacggcttgcaaaactttcagatggagtggctgtgctgaaggttggtgggacaagtgatgttgaagtgaatgaaaagaaagacagagttacagatgcccttaatgctacaagagctgctgttgaagaaggcattgttttgggagggggttgtgccctccttcgatgcattccagccttggactcattgactccagctaatgaagatcaaaaaattggtatagaaattattaaaagaacactcaaaattccagcaatgaccattgctaagaatgcaggtgttgaaggatctttgatagttgagaaaattatgcaaagttcctcagaagttggttatgatgctatggctggagattttgtgaatatggtggaaaaaggaatcattgacccaacaaaggttgtgagaactgctttattggatgctgctggtgtggcctctctgttaactacagcagaagttgtagtcacagaaattcctaaagaagagaaggaccctggaatgggtgcaatgggtggaatgggaggtggtatgggaggtggcatgttctaactcctagactagtgctttacctttattaatgaactgtgacaggaagcccaaggcagtgttcctcaccaataacttcagagaagtcagttggagaaaatgaagaaaaaggctggctgaaaatcactataaccatcagttactggtttcagttgacaaaatatataatggtttactgctgtcattgtccatgcctacagataatttattttgtatttttgaataaaaaacatttgtacattcctgatactgggtacaagagccatgtaccagtgtactgctttcaacttaaatcactgaggcatttttactactattctgttaaaatcaggattttagtgcttgccaccaccagatgagaagttaagcagcctttctgtggagagtgagaataattgtgtacaaagtagagaagtatccaattatgtgacaacctttgtgtaataaaaatttgtttaaagttaaaaaaaaaaaaaaaaaaa

(19) United States
(12) Patent Application Publication
(10) Pub. No.: US 2009/0202618 A1
(43) Pub. Date: Aug. 13, 2009

(54) Dna vaccines encoding hsp60 peptide fragments for treating autoimmune diseases

(75) Inventors: Irun R. Cohen, Rehovot (IL); Francisco Quintana, Capital Federal (AR)

Correspondence Address: WINSTON & STRAWN LLP;PATENT DEPARTMENT, 1700 K STREET, N.W., WASHINGTON, DC, 20006, US

(73) Assignee: Yeda Research & Development Co. Ltd, Revhovot (IL)

(21) Appl. No.: 10/580,176

(22) PCT Filed: Nov. 24, 2004
(86) PCT No.: PCT/IL04/01080

§ 371 Date: Mar. 19, 2007

Related U.S. Application Data
(60) Provisional application No. 60/524,044, filed on Nov. 24, 2003.

(51) Int. Cl. A61K 9/127; C12N 15/00; A61K 39/00; A61K 35/12; C12Q 1/02

(52) U.S. Cl. 424/450; 435/320.1; 424/184.1; 424/185.1; 424/93.2; 435/29

(57) ABSTRACT

The present invention is related to recombinant constructs encoding active fragments of HSP60 which are effective in treating T cell-mediated inflammatory autoimmune diseases by DNA vaccines. The HSP60 fragments of the present invention are identified by their ability to react with T cells isolated from an animal vaccinated with DNA constructs encoding HSP70 to induce Th2/3 T-cell responses.

### FIELD OF THE INVENTION

The present invention relates to HSP60 fragments and to recombinant constructs encoding the active HSP60 fragments effective in preventing or treating T cell-mediated inflammatory autoimmune diseases by DNA vaccination. The present invention further relates to compositions and methods for preventing or treating T cell mediated diseases.

### BACKGROUND OF THE INVENTION

While the normal immune system is closely regulated, aberrations in immune responses are not uncommon. In some instances, the immune system functions inappropriately and reacts to a component of the host as if it were, in fact, foreign. Such response results in an autoimmune disease, in which the host's immune system attacks the host's own tissue. T cells, as the primary regulators of the immune system, directly or indirectly affect such autoimmune pathologies. T cell-mediated autoimmune diseases refer to any condition in which an inappropriate T cell response is a component of the disease. This includes both diseases directly mediated by T cells, and also diseases in which an inappropriate T cell response contributes to the production of abnormal antibodies.

Numerous diseases are believed to result from autoimmune mechanisms. Prominent among these are rheumatoid arthritis, systemic lupus erythematosus, multiple sclerosis, Type I diabetes, myasthenia gravis and pemphigus vulgaris. Autoimmune diseases affect millions of individuals worldwide and the cost of these diseases, in terms of actual treatment expenditures and lost productivity, is measured in billions of dollars annually.

Adjuvant arthritis (AA) is an experimental autoimmune disease that models several features of human rheumatoid arthritis. AA is induced in experimental animals by immunization with heat killed Mycobacterium tuberculosis (Mt) suspended in Incomplete Freund's Adjuvant (IFA). T-cell reactivity against the mycobacterial 65 kDa heat shock protein (HSP65) is involved in the progression of AA. HSP65-specific T-cells directed against an epitope formed by amino acid residues 180-188 (van Eden et al., 1988) cross-react with a self-antigen present in cartilage (van Eden et al., 1985) and can adoptively transfer AA (Holoshitz et al., 1984; Holoshitz et al., 1983). Vaccination with HSP65 or HSP65-peptides can also prevent the development of AA (Billingham et al., 1990; Hogervorst, et al., 1991; Ragno et al., 1997; Moudgil et al., 1997; Anderton et al., 1995; Yang et al., 1992). The regulatory properties of HSP65 in AA are thought to involve the activation of T-cells cross-reactive with the endogenous mammalian 60 kDa heat shock protein (HSP60) (van Eden et al., 2000), which is the mammalian analog of this evolutionally conserved molecule. This hypothesis is supported by the finding that immunization with a recombinant vaccinia virus encoding human HSP60 (about 95% homologous to rat HSP60) prevents (Lopez-Guerrero et al., 1993) or treats (Lopez-Guerrero et al., 1994) AA.

The inventors of the present invention have recently reported that DNA vaccination with DNA encoding human HSP60 prevents AA (Quintana et al., 2002). Protection from AA was associated with the activation of T-cells responding to HSP60 (Quintana et al., 2002). The human HSP60 molecule was formerly designated HSP65, but is now designated HSP60 in view of more accurate molecular weight information; by either designation, the protein is the same. The inventors of the present invention further disclosed that DNA fragments and a regulatory peptide fragment of the human 60-kDa heat shock protein (HSP60) vaccinate against adjuvant arthritis (Quintana et al., 2003).

A preferable method for treating autoimmune diseases includes modulating the immune system of a patient to assist the patient's natural defense mechanisms. Traditional reagents and methods used to attempt to regulate an immune response in a patient also result in unwanted side effects and have limited effectiveness. For example, immunosuppressive reagents (e.g., cyclosporin A, azathioprine, and prednisone) used to treat patients with autoimmune diseases also suppress the patient's entire immune response, thereby increasing the risk of infection. In addition, immunopharmacological reagents used to treat cancer (e.g., interleukins) are short-lived in the circulation of a patient and are ineffective except in large doses.

EP 262710 of Cohen et al. discloses the use of HSP65, or fragments thereof for the preparation of compositions for the alleviation, treatment and diagnosis of autoimmune diseases, especially arthritic conditions. EP 322990 of Cohen et al. discloses that a polypeptide having amino acid sequence 172-192 of HSP65 is capable of inducing resistance to autoimmune arthritis and similar autoimmune diseases. WO 92/04049 of Boog et al. discloses peptides derived from Mycobacterium tuberculosis protein HSP-65 containing at least 7 amino acid residues that inhibit antigen recognition by T lymphocytes in treatment of arthritis and organ rejection.

WO 01/57056 of Karin discloses a method of treating rheumatoid arthritis. The method comprises a step of expressing within the individual at least an immunologically recognizable portion of a cytokine from an exogenous polynucleotide encoding at least a portion of the cytokine, wherein a level of expression of the at least a portion of the cytokine is sufficient to induce the formation of anti-cytokine immunoglobulins which serve for neutralizing or ameliorating the activity of a respective and/or cross reactive endogenous cytokine, to thereby treat rheumatoid arthritis. U.S. Pat. No. 6,316,420 to Karin and coworkers further discloses DNA cytokine vaccines and use of same for protective immunity against multiple sclerosis.

WO 02/16549 of Cohen et al., assigned to the assignee of the present invention, relates to DNA vaccines useful for the prevention and treatment of ongoing autoimmune diseases. The compositions and methods of the invention feature the CpG oligonucleotide, preferably in a motif flanked by two 5′ purines and two 3′ pyrimidines. The vaccines optionally further comprise DNA encoding a peptide or a polypeptide selected from the group consisting of HSP60, p277 or p277 variants. That disclosure is directed to methods and compositions for the ameliorative treatment of ongoing autoimmune disease in general and Insulin Dependent Diabetes Mellitus (IDDM) in particular.

U.S. Pat. No. 5,993,803 discloses that when HSP60, or peptides and analogs thereof, are administered in a recipient subject before transplantation of an organ or tissue, autoimmunity to HSP60 is down-regulated, resulting in the prevention or suppression of graft rejection of the transplanted organ or tissue.

WO 00/27870 of Naparstek and colleagues discloses a series of related peptides derived from heat shock proteins HSP65 and HSP60, their sequences, antibodies, and use as vaccines for conferring immunity against autoimmune and/or inflammatory disorders such as arthritis. These peptides are intended according to that disclosure to represent the shortest sequence or epitope that is involved in protection of susceptible rat strains against adjuvant induced arthritis. These sequences further disclose what the inventors identify as the common “protective motif”.

Due to the medical importance of immune regulation and the inadequacies of existing immunopharmacological reagents, reagents and methods to regulate specific parts of the immune system have been the subject of study for many years.

There exists a long-felt need for an effective means of curing or ameliorating T cell-mediated inflammatory autoimmune diseases. Such a treatment should ideally control the inappropriate T cell response, rather than merely reducing the symptoms.

### SUMMARY OF THE INVENTION

The present invention identifies novel compositions and methods for the treatment of T cell mediated diseases and symptoms. The present invention provides compositions comprising the novel fragments of heat shock protein 60 (HSP60) useful for the treatment or prevention of T cell mediated diseases. In particular, the present invention provides compositions comprising DNA constructs encoding active fragments of HSP60, useful for DNA vaccination to prevent or treat T cell-mediated inflammatory diseases. These constructs are novel in that they encode HSP60 fragments identified by their reaction with T cells previously sensitized to HSP70.

DNA vaccination provides an unexpectedly effective means of expressing antigen in vivo for the generation of both humoral and cellular immune responses. The present invention utilizes this technology to elicit protective immunity against T cell-mediated autoimmune diseases using DNA constructs encoding active fragments of heat shock protein 60 (HSP60).

The inventors have unexpectedly discovered the existence of an immunological inter-relationship, or cross talk, between different heat shock proteins (HSPs) in eliciting protective immunity against T cell-mediated autoimmune diseases. The novel HSP60 fragments are capable of inducing Th2/3 T-cell responses associated with the arrest of experimental arthritis specifically in cells isolated from animals vaccinated with DNA constructs encoding HSP70. As disclosed herein for the first time, lymph node cells (LNC) from pHSP70-vaccinated rats exposed to specific HSP60 fragments exhibited increased secretion of IL-10 and TGFβ1 and decreased secretion of IFNγ. These altered patterns of response are considered beneficial in arresting deleterious autoimmune reactions.

In one aspect, the present invention provides DNA vaccines encoding HSP60 fragments for preventing or treating T cell-mediated inflammatory autoimmune diseases. The HSP60 fragments of the present invention are characterized in that they react with T cells isolated from an animal vaccinated with DNA constructs encoding HSP70 to induce Th2/3 T-cell responses. The HSP60 fragments are derived from mammalian, preferably human HSP60. However, HSP60 fragments derived from other mammalian HSP60 are within the scope of the present invention.

Preferred fragments of human HSP60 (SEQ ID NO:14) correspond to amino acids 271-290 (SEQ ID NO:1), amino acids 346-365 (SEQ ID NO:2), amino acids 361-380 (SEQ ID NO:3), amino acids 391-410 (SEQ ID NO:4), amino acids 406-425 (SEQ ID NO:5), amino acids 436-455 (SEQ ID NO:6), amino acids 466-485 (SEQ ID NO:7), amino acids 481-500 (SEQ ID NO:8) or amino acids 496-515 (SEQ ID NO:9). It is noted that both shorter active fragments derived from the peptides denoted as SEQ ID NOS:1-9 and longer peptides comprising these sequences are within the scope of the present invention. The HSP60 fragments according to the present invention are preferably 7-30 amino acids in length.

The compositions and methods of the present invention are effective in many T-cell mediated inflammatory autoimmune diseases including but not limited to: rheumatoid arthritis, collagen II arthritis, multiple sclerosis, autoimmune neuritis, systemic lupus erythematosus, psoriasis, juvenile onset diabetes, Sjogren's disease, thyroid disease, sarcoidosis, autoimmune uveitis, inflammatory bowel disease (Crohn's and ulcerative colitis) and autoimmune hepatitis.

The treatment with the DNA vaccines of the present invention provides long-term expression of the active HSP60 fragments ranging from several days to several months. Such long-term expression allows for the maintenance of an effective dose of the encoded fragments sufficient to prevent or treat the disease with few or no side effects. The use of DNA vaccines limits the frequency of administration of the pharmaceutical composition needed to treat a subject. In addition, because of the lack of side effects in the host, the pharmaceutical compositions of the present invention can be used in repeated treatments.

In another aspect, the present invention provides novel recombinant constructs comprising a nucleic acid sequence encoding an HSP60 fragment according to the present invention, being operatively linked to at least one transcription control element. The HSP60 fragments according to the present invention are preferably derived from human HSP60, however other mammalian HSP60 fragments are within the scope of the present invention.

In a preferred embodiment, the recombinant constructs encode expressible active fragments of human HSP60 (SEQ ID NO:14), said active fragments selected from: amino acids 271-290 (SEQ ID NO:1), amino acids 346-365 (SEQ ID NO:2), amino acids 361-380 (SEQ ID NO:3), amino acids 391-410 (SEQ ID NO:4), amino acids 406-425 (SEQ ID NO:5), amino acids 436-455 (SEQ ID NO:6), amino acids 466-485 (SEQ ID NO:7), amino acids 481-500 (SEQ ID NO:8) and amino acids 496-515 (SEQ ID NO:9). It is noted that both shorter active fragments derived from the peptides denoted as SEQ ID NOS:1-9 and longer peptides comprising these sequences are within the scope of the present invention.

According to various specific embodiments, the constructs of the present invention comprise at least one transcription control element selected from the group consisting of: RSV control sequences, CMV control sequences, retroviral LTR sequences, SV-40 control sequences and β-actin control sequences.

In another aspect, the present invention provides an eukaryotic expression vector comprising the recombinant constructs of the present invention. According to various embodiments, the eukaryotic expression vector is selected from pcDNA3, pCR3.1, pcDNA3.1(+/−), pZeoSV2(+/−), pSecTag2, pDisplay, pEF/myc/cyto, pCMV/myc/cyto, pCI, pBK-RSV, pBK-CMV and pTRES.

Another aspect of the present invention provides a pharmaceutical composition effective for preventing or treating a T cell-mediated inflammatory autoimmune disease, the composition comprising (a) a recombinant construct comprising an isolated nucleic acid sequence encoding a fragment of HSP60 according to the present invention, the nucleic acid sequence being operatively linked to one or more transcription control sequences; and (b) a pharmaceutically acceptable carrier.

The pharmaceutical compositions comprising the recombinant constructs according to the present invention may advantageously comprise liposomes, micelles, emulsions or cells. Still further embodiments utilize a virus as is known in the art in order to introduce and express the nucleic acid sequences according to the present invention in the host cells.

In another aspect, the present invention is related to a method of inhibiting or preventing the symptoms of a T-cell mediated inflammatory autoimmune disease, the method comprising administering to a subject in need of such treatment, preferably a human subject, a pharmaceutical composition comprising a recombinant construct, said recombinant construct comprising an isolated nucleic acid sequence encoding a fragment of HSP60 according to the present invention, thereby inhibiting or preventing the symptoms of said autoimmune disease.

According to various embodiments, the compositions and methods of the present invention are effective in T-cell mediated inflammatory autoimmune diseases including but not limited to: rheumatoid arthritis, collagen II arthritis, multiple sclerosis, autoimmune neuritis, systemic lupus erythematosus, psoriasis, juvenile onset diabetes, Sjogren's disease, thyroid disease, sarcoidosis, autoimmune uveitis, inflammatory bowel disease (Crohn's and ulcerative colitis) and autoimmune hepatitis.

The present invention is particularly exemplified by the animal disease model of adjuvant arthritis (AA), a T cell-mediated autoimmune disease that serves as an experimental model for rheumatoid arthritis. This model is intended as a non-limitative example used for illustrative purposes of the principles of the invention.

In one embodiment, the pharmaceutical composition of the present invention is administered to a subject at risk of developing a T-cell mediated inflammatory autoimmune disease, thus serving as a preventive treatment. In another embodiment, the pharmaceutical composition of the present invention is administered to a subject during the initial stages of the disease or after the appearance of disease symptoms.

According to another aspect, the present invention provides a method for preventing or treating a T cell-mediated inflammatory autoimmune disease comprising the steps of (a) obtaining cells from a subject; (b) transfecting the cells ex vivo with a recombinant construct comprising an isolated nucleic acid sequence encoding an HSP60 fragment according to the present invention, the nucleic acid sequence being operatively linked to one or more transcription control sequences; and (c) reintroducing the transfected cells to the subject. The cells obtained from the subject for ex-vivo transfection are preferably T cells, however, the use of other cells for ex-vivo transfection is within the scope of the present invention.

According to another aspect, the present invention provides a method for preventing or treating a T cell-mediated inflammatory autoimmune disease comprising the steps of (a) obtaining cells from a subject; (b) infecting the cells ex vivo with a virus comprising a recombinant construct comprising an isolated nucleic acid sequence encoding an HSP60 fragment according to the present invention, the nucleic acid sequence being operatively linked to one or more transcription control sequences; and (c) reintroducing the infected cells to the subject. The cells obtained from the subject for ex-vivo infection with a virus are preferably T cells, however, the use of other cells for ex-vivo infection is within the scope of the present invention.

According to another aspect, the present invention provides a method for preventing or treating a T cell-mediated inflammatory autoimmune disease comprising administering to a subject in need thereof a pharmaceutical composition comprising (a) a fragment of mammalian HSP60 according to the present invention capable of inducing Th2/3 T-cell responses in T cells isolated from an animal vaccinated with DNA constructs encoding HSP70; and (b) a pharmaceutically acceptable carrier. Preferred HSP60 fragments are human HSP60 fragments having an amino acid sequence as set forth in any one of SEQ ID NO:1 through SEQ ID NO:9. It is noted that both shorter active fragments derived from the peptides denoted as SEQ ID NOS:1-9 and longer peptides comprising these sequences are within the scope of the present invention.

According to another aspect, the present invention provides a method of preventing or treating arthritis, said method comprising administering to a subject in need thereof a pharmaceutical composition comprising (a) a fragment of mammalian HSP60 according to the present invention; and (b) a pharmaceutically acceptable carrier, thereby preventing or treating arthritis. According to various embodiments, the carrier comprises a delivery vehicle that delivers the fragment to the subject. Preferred HSP60 fragments are human HSP60 fragments having an amino acid sequence as set forth in any one of SEQ ID NO:1 through SEQ ID NO:9. It is noted that both shorter active fragments derived from the peptides denoted as SEQ ID NOS:1-9 and longer peptides comprising these sequences are within the scope of the present invention.

The pharmaceutical composition of the present invention may be administered according to known modes for peptide or nucleic acid administration, including oral, intravenous, subcutaneous, intraarticular, intramuscular, inhalation, intranasal, intrathecal, intradermal, transdermal or other known routes.

According to another aspect, the present invention provides a method of preventing or treating arthritis, said method comprising the steps of (a) obtaining T cells from a subject; (b) contacting the T cells ex vivo with an active amount of a fragment of HSP60 according to the present invention; and (c) reintroducing the treated T cells to the subject, thereby preventing or treating arthritis. Preferred HSP60 fragments are human HSP60 fragments having an amino acid sequence as set forth in any one of SEQ ID NO:1 through SEQ ID NO:9. It is noted that both shorter active fragments derived from the peptides denoted as SEQ ID NOS:1-9 and longer peptides comprising these sequences are within the scope of the present invention.

According to another aspect, the present invention provides a method of screening for active fragments of HSP60 capable of inducing Th2/3 T-cell responses. The method comprising: (a) administrating a DNA construct encoding HSP70 to an animal in a sufficient amount to induce expression of HSP70 in the animal; (b) obtaining T cells from said animal; (c) contacting the cells with a candidate HSP60 fragment for sufficient time for inducing cytokine secretion in said cells, and (d) determining the secretion of IL-10, TGFβ1 and IFNγ from said cells, wherein if the secretion of IL-10 and TGFβ1 is increased and the secretion of IFNγ is decreased than the candidate HSP60 fragment is capable of inducing Th2/3 T-cell responses.

These and further embodiments will be apparent from the detailed description and examples that follow.

### BRIEF DESCRIPTION OF THE DRAWINGS

FIG. 1 demonstrates the In vitro expression of pHSP70 and pHSP90 vectors in the presence of [³⁵S]-methionine. The newly synthesized proteins were analysed by PAGE-SDS followed by autoradiography.

FIG. 2 demonstrates the induction of specific immune responses in pHSP70 or pHSP90-vaccinated rats. IgG antibodies to HSP70 (A) or OVA (B) were studied in pcDNA3- or pHSP70-vaccinated rats; antibodies to GST-HSP90 (C) and GST (D) were studied in pcDNA3- or pHSP90-vaccinated rats.

FIG. 3 demonstrates the inhibition of AA by vaccination with pHSP70 and pHSP90. (A) Time course of AA. Lewis rats were vaccinated with pHSP70, pHSP90 or pcDNA3 as described in FIG. 2, AA was induced, and arthritis scores were assessed every two or three days starting at day 10. (B) Leg swelling measured at day 26 after AA induction.

FIG. 4 demonstrates the T-cell proliferation in response to Mt-derived antigens in LNC collected from pHSP70 or pHSP90-vaccinated rats. Lewis rats were vaccinated with pHSP70, pHSP90 or pcDNA3 as described in FIG. 2 and AA was induced. Twenty-six days later LNC were collected and the proliferative responses to PPD (A) or HSP65, Mt176-90 and HSP71 (B) were studied.

FIG. 5 demonstrates the cytokine secretion in response to Mt-derived antigens in LNC collected from pHSP70 or pHSP90-vaccinated rats. Lewis rats were vaccinated with pHSP70, pHSP90 or pcDNA3 as described in FIG. 2 and AA was induced. Twenty-six days later LNC were prepared, stimulated in vitro for 72 hr with PPD, HSP71, HSP65 and Mt176-190, and the supernatants were tested for the amounts of secreted (A) IFNγ, (B) IL-10 or (C) TGFβ1.

FIG. 6 demonstrates the T-cell proliferation in response to HSP70, HSP90 and HSP60 in LNC collected from pHSP70 or pHSP90-vaccinated rats. Lewis rats were vaccinated with pHSP70, pHSP90 or pcDNA3 as described in FIG. 2 and AA was induced. Twenty-six days later, LNC were collected, and the proliferative responses to HSP70, HSP90 and HSP60 were studied.

FIG. 7 demonstrates the cytokine secretion in response to HSP70, HSP90 and HSP60 in LNC collected from pHSP70 or pHSP90-vaccinated rats. Lewis rats were vaccinated with pHSP70, pHSP90 or pcDNA3 as described in FIG. 2 and AA was induced. Twenty-six days later LNC were prepared, stimulated in vitro for 72 hr with HSP70, HSP90 and HSP60, and the supernatants were tested for the amounts of secreted (A) IFNγ, (B) IL-10 or (C) TGFβ1.

FIG. 8 demonstrates that pHSP60 and pHSP65-vaccination activates in vitro T-cell proliferation in response to HSP70 and HSP71. Lewis rats were vaccinated with pHSP60, pHSP65 or pcDNA3 as described in FIG. 2 and AA was induced. Twenty-six days later, LNC were collected, and the proliferative responses to HSP70 and HSP71 were studied.

FIG. 9 demonstrates that pHSP60 and pHSP65-vaccination activate cytokine secretion in vitro in response to HSP70 and HSP71. Lewis rats were vaccinated with pHSP60, pHSP65 or pcDNA3 as described in FIG. 2 and AA was induced. Twenty-six days later, LNC were prepared and stimulated in vitro for 72 hr with HSP70 or HSP71 and the supernatants were tested for the amounts of secreted (A) IFNγ, (B) IL-10 or (C) TGFβ1.

FIG. 10 demonstrates that pHSP70 vaccination up-regulates serum HSP60. Lewis rats were vaccinated with pHSP60 or pcDNA3 as described in FIG. 2 and AA was induced. 26 days later, blood samples were collected and serum HSP60 was quantified. Mean HSP60 levels (±SEM) in serum. *p<0.05 compared with the pcDNA3 group.

### DETAILED DESCRIPTION OF THE INVENTION

According to the present invention it is now disclosed that it is possible to treat or prevent T cell-mediated inflammatory autoimmune diseases by using DNA vaccines encoding active fragments of HSP60 that react with T cells isolated from an animal vaccinated with DNA constructs encoding HSP70 to induce Th2/3 T-cell responses.

The present invention is based in part on studies of the role of DNA vaccines encoding HSP60 fragments in adjuvant-induced arthritis in experimental rats. The present invention is based on the unexpected discovery that specific HSP60 fragments are capable of inducing Th2/3 T-cell responses associated with the arrest of experimental arthritis specifically in cells isolated from animals vaccinated with DNA constructs encoding HSP70, as disclosed herein for the first time. Specifically, lymph node cells (LNC) from pHSP70-vaccinated rats exposed to specific HSP60 fragments exhibited increased secretion of IL-10 and TGFβ1 and decreased secretion of IFNγ.

The present invention is based in part on studies of the role of the immune response to HSP60 in adjuvant-induced arthritis in experimental rats, using DNA vaccines encoding HSP60 fragments. The results led to the identification of novel constructs encoding fragments of the HSP60 sequence that could effectively suppress AA. Surprisingly, immunization with pHSP70 or pHSP90 induced a T-cell response to HSP60. However, the T-cell epitopes targeted by pHSP70-vaccinated rats were different than those targeted by pHSP60 vaccinated rats: HSP60 DNA vaccination induces a response to the Hu3 peptide alone, while HSP70 DNA vaccination induces responses to several HSP60 peptides, but not to Hu3 (Table I).

Thus, the results led to the identification of novel fragments of human HSP60 (SEQ ID NO:14), useful for treating or ameliorating symptoms of autoimmune diseases, namely: amino acids 361-380 (SEQ ID NO:3), amino acids 391-410 (SEQ ID NO:4), amino acids 406-425 (SEQ ID NO:5), and amino acids 496-515 (SEQ ID NO:9).

The results also led to the identification of novel uses for known peptides of HSP60 in treating autoimmune diseases other than insulin-dependent diabetes mellitus (IDDM), specifically exemplified as useful for arthritis. HSP60 fragments comprising amino acids 271-290 (SEQ ID NO:1), amino acids 346-365 (SEQ ID NO:2), amino acids 436-455 (SEQ ID NO:6), amino acids 466-485 (SEQ ID NO:7), and amino acids 481-500 (SEQ ID NO:8) were previously implicated in the diagnosis and treatment of insulin-dependent diabetes mellitus (WO 97/01959), and were herein unexpectedly found to react with T cells sensitized to HSP70.

HSP60, HSP70 and HSP90 share no sequence homology and are not immunologically cross-reactive. One possible explanation for the induction of HSP60-specific T-cell responses by pHSP70 or pHSP90 is self-vaccination with endogenous self-HSP60 induced and/or released as a result of DNA vaccination. Without wishing to be bound by any particular hypothesis or mechanism of action, this hypothesis is supported by the detection of increased levels of circulating HSP60 in pHSP70-vaccinated rats. In addition, vaccination with pHSP60 induced a T-cell response to HSP70.

The term “Th2/3 T-cell responses” refers to increased secretion of IL-10 and TGFβ1 and decreased secretion of IFNγ by T cells considered to be beneficial in arresting deleterious autoimmune reactions.

The term “T-cell mediated autoimmune disease” refers to any condition in which an inappropriate T cell response is a component of the disease. The term is intended to include both diseases directly mediated by T cells, and also diseases in which an inappropriate T cell response contributes to the production of abnormal antibodies.

The compositions and methods of the present invention are effective in many T-cell mediated inflammatory autoimmune diseases, including but not limited to: rheumatoid arthritis, collagen II arthritis, multiple sclerosis, autoimmune neuritis, systemic lupus erythematosus, psoriasis, juvenile onset diabetes, Sjogren's disease, thyroid disease, sarcoidosis, autoimmune uveitis, inflammatory bowel disease (Crohn's and ulcerative colitis) and autoimmune hepatitis.

The present invention provides an effective method of DNA vaccination for T cell-mediated inflammatory autoimmune diseases, which avoids many of the problems associated with the previously suggested methods of treatment. By vaccinating, rather than passively administering heterologous antibodies, the host's own immune system is mobilized to suppress the autoaggressive T cells. Thus, the suppression is persistent and may involve any and all immunological mechanisms in effecting that suppression. This multi-faceted response is more effective than the uni-dimensional suppression achieved by passive administration of monoclonal antibodies or extant-derived regulatory T cell clones.

In one aspect, the present invention is related to novel recombinant constructs comprising a nucleic acid sequence corresponding to HSP60 fragments, the nucleic acid sequence being operatively linked to at least one transcription control element. Preferably, the recombinant constructs of the present invention correspond to human HSP60 fragments. However, recombinant constructs corresponding to the rat or mouse HSP60 may also be used in the present invention.

In a preferred embodiment, the recombinant constructs encode expressible active fragments of human HSP60 (SEQ ID NO:14), said active fragments selected from: amino acids 271-290 (SEQ ID NO:1), amino acids 346-365 (SEQ ID NO:2), amino acids 361-380 (SEQ ID NO:3), amino acids 391-410 (SEQ ID NO:4), amino acids 406-425 (SEQ ID NO:5), amino acids 436-455 (SEQ ID NO:6), amino acids 466-485 (SEQ ID NO:7), amino acids 481-500 (SEQ ID NO:8) and amino acids 496-515 (SEQ ID NO:9). It is noted that both shorter active fragments derived from the peptides denoted as SEQ ID NOS:1-9 and longer peptides comprising these sequences are within the scope of the present invention.

For the preparation of recombinant constructs, nucleic acid sequences encoding the HSP60 fragments of the invention may be synthesized according to the native nucleic acid sequence of HSP60 (SEQ ID NO:15). In addition, it will be appreciated by those skilled in the art that as a result of the degeneracy of the genetic code, a multitude of nucleotide sequences encoding the HSP60 fragments of the invention, some bearing minimal homology to the nucleotide sequences of the naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence of naturally occurring HSP60 fragments of the invention, and all such variations are to be considered as being specifically disclosed.

The nucleic acid sequence corresponding to mammalian heat shock proteins may include DNA, RNA, or derivatives of either DNA or RNA. An isolated nucleic acid sequence encoding heat shock proteins can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologues thereof, including, but not limited to, natural allelic variants and modified nucleic acid sequences in which nucleotides have been inserted, deleted, substituted, and/or inverted in such a manner that such modifications do not substantially interfere with the nucleic acid molecule's ability to encode a functional heat shock protein or an active fragment thereof.

A nucleic acid sequence homologue can be produced using a number of methods known to those skilled in the art (see, for example, Sambrook et al., 1989). For example, nucleic acid sequences can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid.

The present invention includes a nucleic acid sequence operatively linked to one or more transcription control sequences to form a recombinant molecule. The phrase “operatively linked” refers to linking a nucleic acid sequence to a transcription control sequence in a manner such that the molecule is able to be expressed when transfected (i.e., transformed, transduced or transfected) into a host cell. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in animal, bacteria, helminth, insect cells, and preferably in animal cells. More preferred transcription control sequences include, but are not limited to RSV control sequences, CMV control sequences, retroviral LTR sequences, SV-40 control sequences and β-actin control sequences as well as other sequences capable of controlling gene expression in eukaryotic cells. Additional suitable transcription control sequences include tissue-specific promoters and enhancers (e.g., T cell-specific enhancers and promoters). Transcription control sequences of the present invention can also include naturally occurring transcription control sequences naturally associated with a gene encoding a heat shock protein of the present invention.

The present invention is further related to an expression vector comprising the recombinant constructs of the present invention. Suitable eukaryotic expression vector is for example: pcDNA3, pcDNA3.1(+/−), pZeoSV2(+/−), pSecTag2, pDisplay, pEF/myc/cyto, pCMV/myc/cyto, pCR3.1, pCI, pBK-RSV, pBK-CMV, pTRES or their derivatives.

According to the present invention, a host cell can be transfected in vivo (i.e., in an animal) or ex vivo (i.e., outside of an animal). Transfection of a nucleic acid molecule into a host cell can be accomplished by any method by which a nucleic acid molecule can be inserted into the cell. Transfection techniques include, but are not limited to, transfection, electroporation, microinjection, lipofection, adsorption, and protoplast fusion. Preferred methods to transfect host cells in vivo include lipofection and adsorption.

A host cell may also be infected in vivo or ex vivo by a viral vector comprising the nucleic acid molecules of the present invention. A viral vector includes an isolated nucleic acid molecule useful in the present invention, in which the nucleic acid molecules are packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses (AAV) and retroviruses. Recombinant adenoviruses have several advantages over retroviral and other viral-based gene delivery methods. Adenoviruses have never been shown to induce tumors in humans and have been safely used as live vaccines. Adenovirus does not integrate into the human genome as a normal consequence of infection, thereby greatly reducing the risk of insertional mutagenesis possible with retrovirus or AAV vectors. This lack of stable integration also leads to an additional safety feature in that the transferred gene effect will be transient, as the extra-chromosomal DNA will be gradually lost with continued division of normal cells. Stable, high titer recombinant adenovirus can be produced at levels not achievable with retrovirus or AAV, allowing enough material to be produced to treat a large patient population.

It may be appreciated by one skilled in the art that use of recombinant DNA technologies can improve expression of transfected nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, and deletion of sequences that destabilize transcripts.

According to yet another aspect of the present invention there is provided a pharmaceutical composition suitable for effecting the above methods of the present invention. The composition includes a recombinant construct including an isolated nucleic acid sequence encoding an active HSP60 fragment according to the present invention, the nucleic acid sequence being operatively linked to one or more transcription control sequences, and a pharmaceutically acceptable carrier.

The composition according to the present invention is useful for treating many T cell-mediated inflammatory autoimmune diseases, including but not limited to multiple sclerosis, rheumatoid arthritis, collagen II arthritis, autoimmune neuritis, systemic lupus erythematosus, psoriasis, juvenile onset diabetes, Sjogren's disease, thyroid disease, sarcoidosis, autoimmune uveitis, inflammatory bowel disease (Crohn's and ulcerative colitis) and autoimmune hepatitis.

The pharmaceutical composition of the invention is administered to a subject in need of said treatment. According to still further features in the described preferred embodiments the subject is selected from the group consisting of humans, dogs, cats, sheep, cattle, horses and pigs.

The pharmaceutical composition of the present invention further comprises a pharmaceutically acceptable carrier. As used herein, a “carrier” refers to any substance suitable as a vehicle for delivering a nucleic acid sequence of the present invention to a suitable in vivo site. As such, carriers can act as a pharmaceutically acceptable excipient of a pharmaceutical composition containing a nucleic acid molecule of the present invention. Preferred carriers are capable of maintaining a nucleic acid molecule of the present invention in a form that, upon arrival of the nucleic acid molecule to a cell, the nucleic acid molecule is capable of entering the cell and being expressed by the cell.

Carriers of the present invention include: (1) excipients or formularies that transport, but do not specifically target a nucleic acid molecule to a cell (referred to herein as non-targeting carriers); and (2) excipients or formularies that deliver a nucleic acid molecule to a specific site in an animal or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- and o-cresol, formalin and benzol alcohol. Preferred auxiliary substances for aerosol delivery include surfactant substances non-toxic to an animal, for example, esters or partial esters of fatty acids containing from about six to about twenty-two carbon atoms. Examples of esters include, caproic, octanoic, lauric, palmitic, stearic, linoleic, linolenic, olesteric, and oleic acids. Other carriers can include metal particles (e.g., gold particles) for use with, for example, a biolistic gun through the skin. Pharmaceutical compositions of the present invention can be sterilized by conventional methods.

Targeting carriers are herein referred to as “delivery vehicles”. Delivery vehicles of the present invention are capable of delivering a pharmaceutical composition of the present invention to a target site in an animal. A “target site” refers to a site in an animal to which one desires to deliver a pharmaceutical composition. Examples of delivery vehicles include, but are not limited to, artificial and natural lipid-containing delivery vehicles. Natural lipid-containing delivery vehicles include cells and cellular membranes. Artificial lipid-containing delivery vehicles include liposomes and micelles.

A delivery vehicle of the present invention can be modified to target to a particular site in an animal, thereby targeting and making use of a nucleic acid molecule of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

According to one embodiment, fat emulsions may be used as a vehicle for DNA vaccines. Two examples of such emulsions are the available commercial fat emulsions known as Intralipid and Lipofundin. “Intralipid” is a registered trademark of Kabi Pharmacia, Sweden, for a fat emulsion for intravenous nutrition, described in U.S. Pat. No. 3,169,094. “Lipofundin” is a registered trademark of B. Braun Melsungen, Germany. Both contain soybean oil as fat (100 or 200 g in 1,000 ml distilled water: 10% or 20%, respectively). Egg-yolk phospholipids are used as emulsifiers in Intralipid (12 g/l distilled water) and egg-yolk lecithin in Lipofundin (12 g/l distilled water). Isotonicity results from the addition of glycerol, (25 g/l) both in Intralipid and Lipofundin.

According to another embodiment, the delivery vehicle of the present invention may be a liposome. A liposome is capable of remaining stable in an animal for a sufficient amount of time to deliver a nucleic acid sequence of the present invention to a preferred site in the animal. A liposome of the present invention is preferably stable in the animal into which it has been administered for at least about 30 minutes, more preferably for at least about 1 hour and even more preferably for at least about 24 hours.

A liposome of the present invention comprises a lipid composition that is capable of fusing with the plasma membrane of the targeted cell to deliver a nucleic acid molecule into a cell. Preferably, the transfection efficiency of a liposome of the present invention is about 0.5 microgram (μg) of DNA per 16 nanomole (nmol) of liposome delivered to about 10⁶ cells, more preferably about 1.0 μg of DNA per 16 nmol of liposome delivered to about 10⁶ cells, and even more preferably about 2.0 μg of DNA per 16 nmol of liposome delivered to about 10⁶ cells.

A preferred liposome of the present invention is between about 100 and 500 nanometers (nm), more preferably between about 150 and 450 nm and even more preferably between about 200 and 400 nm in diameter.

Suitable liposomes for use with the present invention include any liposome. Preferred liposomes of the present invention include those liposomes standardly used in, for example, gene delivery methods known to those of skill in the art. More preferred liposomes comprise liposomes having a polycationic lipid composition and/or liposomes having a cholesterol backbone conjugated to polyethylene glycol.

Complexing a liposome with a nucleic acid sequence of the present invention can be achieved using methods standard in the art. A suitable concentration of a nucleic acid molecule of the present invention to add to a liposome includes a concentration effective for delivering a sufficient amount of nucleic acid molecule to a cell such that the cell can produce sufficient heat shock protein to regulate effector cell immunity in a desired manner. Preferably, from about 0.1 μg to about 10 μg of nucleic acid sequence of the present invention is combined with about 8 nmol liposomes, more preferably from about 0.5 μg to about 5 μg of nucleic acid molecule is combined with about 8 nmol liposomes, and even more preferably about 1.0 μg of nucleic acid molecule is combined with about 8 nmol liposomes.

According to another embodiment, the delivery vehicle comprises a recombinant cell vaccine. Preferred recombinant cell vaccines of the present invention include cell vaccines, in which allogeneic (i.e., cells derived from a source other than a patient, but that are histiotype compatible with the patient) or autologous (i.e., cells isolated from a patient) cells are transfected with recombinant molecules contained in a pharmaceutical composition, irradiated and administered to a patient by, for example, intradermal, intravenous or subcutaneous injection. Pharmaceutical compositions to be administered by cell vaccine, include recombinant molecules of the present invention without carrier.

In order to treat a subject with disease, a pharmaceutical composition of the present invention is administered to the subject in an effective manner such that the composition is capable of treating that subject from disease. For example, a recombinant molecule, when administered to a subject in an effective manner, is able to stimulate effector cell immunity in a manner that is sufficient to alleviate the disease afflicting the subject.

According to the present invention, treatment of a disease refers to alleviating a disease and/or preventing the development of a secondary disease resulting from the occurrence of a primary disease. An effective administration protocol (i.e., administering a pharmaceutical composition in an effective manner) comprises suitable dose parameters and modes of administration that result in treatment of a disease. Effective dose parameters and modes of administration can be determined using methods standard in the art for a particular disease. Such methods include, for example, determination of survival rates, side effects (i.e., toxicity) and progression or regression of disease.

In accordance with the present invention, a suitable single dose size is a dose that is capable of treating a subject with disease when administered one or more times over a suitable time period. Doses can vary depending upon the disease being treated. Doses of a pharmaceutical composition of the present invention suitable for use with direct injection techniques can be used by one of skill in the art to determine appropriate single dose sizes for systemic administration based on the size of a subject. A suitable single dose of a pharmaceutical composition is a sufficient amount of the HSP60 fragment-encoding recombinant sequence to reduce, and preferably eliminate, the T-cell mediated autoimmune disease following transfection of the recombinant molecules into cells. A preferred single dose of heat shock protein-encoding recombinant molecule is an amount that, when transfected into a target cell population leads to the production of from about 250 fentograms (fg) to about 1 μg, preferably from about 500 fg to about 500 picogram (pg), and more preferably from about 1 pg to about 100 pg of a heat shock protein or fragment thereof per transfected cell.

A preferred single dose of heat shock protein-encoding recombinant molecule complexed with liposomes, is from about 100 μg of total DNA per 800 nmol of liposome to about 2 mg of total recombinant molecules per 16 micromole (μmol) of liposome, more preferably from about 150 μg per 1.2 μmol of liposome to about 1 mg of total recombinant molecules per 8 μmol of liposome, and even more preferably from about 200 μg per 2 μmol of liposome to about 400 μg of total recombinant molecules per 3.2 μmol of liposome.

A preferred single dose of heat shock protein-encoding recombinant molecule in a non-targeting carrier to administer to a subject, is from about 100 μg to about 4 mg of total recombinant molecules, more preferably from about 150 μg to about 3 mg of total recombinant molecules, and even more preferably from about 200 μg to about 2 mg of total recombinant molecules.

It will be obvious to one of skill in the art that the number of doses administered to a subject is dependent upon the extent of the disease and the response of an individual patient to the treatment. Thus, it is within the scope of the present invention that a suitable number of doses includes any number required to cause regression of a disease. A preferred protocol is monthly administrations of single doses (as described above) for up to about 1 year. A preferred number of doses of a pharmaceutical composition comprising heat shock protein-encoding recombinant molecule in a non-targeting carrier or complexed with liposomes is from about 1 to about 10 administrations per patient, preferably from about 2 to about 8 administrations per patient, and even more preferably from about 3 to about 5 administrations per patient. Preferably, such administrations are given once every 2 weeks until signs of remission appear, then once a month until the disease is gone.

A pharmaceutical composition is administered to a subject in a fashion to enable expression of the administered recombinant molecule of the present invention into a curative protein in the subject to be treated for disease. A pharmaceutical composition can be administered to a subject in a variety of methods including, but not limited to, local administration of the composition into a site in a subject, and systemic administration.

Pharmaceutical compositions to be delivered by local administration may be selected from: (a) recombinant molecules of the present invention in a non-targeting carrier (e.g., as “naked” DNA molecules, such as is taught, for example in Wolff et al., 1990); and (b) recombinant molecules of the present invention complexed to a delivery vehicle of the present invention. Suitable delivery vehicles for local administration comprise liposomes or emulsions. Delivery vehicles for local administration may further comprise ligands for targeting the vehicle to a particular site.

Pharmaceutical compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site. Systemic administration is particularly advantageous when organs, in particular difficult to reach organs (e.g., heart, spleen, lung or liver) are the targeted sites of treatment.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., 1992, which is incorporated herein by reference in its entirety). Oral delivery can be performed by complexing a pharmaceutical composition of the present invention to a carrier capable of withstanding degradation by digestive enzymes in the gut of an animal. Examples of such carriers, include plastic capsules or tablets, such as those known in the art. Topical delivery can be performed by mixing a pharmaceutical composition of the present invention with a lipophilic reagent (e.g., DMSO) that is capable of passing into the skin.

According to various embodiments, suitable doses, single dose sizes, number of doses and modes of administration of a pharmaceutical composition of the present invention, useful in a treatment method of the present invention, are disclosed in detail herein. Alternative regimes, doses and the like are within the skill of the medical practitioner as required, taking into consideration the condition and subject to be treated.

A pharmaceutical composition of the present invention is advantageous for the treatment of autoimmune diseases in that the composition suppresses the harmful stimulation of T cells by autoantigens (i.e., a “self”, rather than a foreign antigen). DNA constructs encoding HSP60 fragments in a pharmaceutical composition, upon transfection into a cell, produce HSP60 fragments that reduce the harmful activity of T cells involved in an autoimmune disease. A preferred pharmaceutical composition for use in the treatment of autoimmune disease comprises HSP60 fragment-encoding recombinant molecule combined with a non-targeting carrier of the present invention, preferably saline or phosphate buffered saline.

A single dose of heat shock protein-encoding nucleic acid molecule in a non-targeting carrier to administer to a subject to treat an autoimmune disease is preferably from about 0.1 μg to about 200 μg of total recombinant molecules per kilogram (g) of body weight, more preferably from about 0.5 μg to about 150 μg of total recombinant molecules per kg of body weight, and even more preferably from about 1 μg to about 10 μg of total recombinant molecules per kg of body weight.

The number of doses of heat shock protein-encoding recombinant molecule in a non-targeting carrier to be administered to a subject to treat an autoimmune disease is preferably an injection about once every 6 months, more preferably about once every 3 months, and even more preferably about once a month.

A preferred method to administer a pharmaceutical composition of the present invention to treat an autoimmune disease is by direct injection. Direct injection techniques are particularly important in the treatment of an autoimmune disease. Preferably, a pharmaceutical composition is injected directly into muscle cells in a patient, which results in prolonged expression (e.g., weeks to months) of a recombinant molecule of the present invention. Preferably, a recombinant molecule of the present invention in the form of “naked DNA” is administered by direct injection into muscle cells in a patient.

According to another aspect, the present invention provides a method for preventing or treating a T cell-mediated inflammatory autoimmune disease comprising administering to a subject in need thereof a pharmaceutical composition comprising (a) a fragment of mammalian HSP60 according to the present invention capable of inducing Th2/3 T-cell responses in T cells isolated from an animal vaccinated with DNA constructs encoding HSP70; and (b) a pharmaceutically acceptable carrier. Preferred HSP60 fragments are human HSP60 fragments having an amino acid sequence as set forth in any one of SEQ ID NO:1 through SEQ ID NO:9. It is noted that both shorter active fragments derived from the peptides denoted as SEQ ID NOS:1-9 and longer peptides comprising these sequences are within the scope of the present invention.

Preferred peptides according to the present invention may be synthesized using any method known in the art, including peptidomimetic methodologies. These methods include solid phase as well as solution phase synthesis methods. The conjugation of the peptidic and permeability moieties may be performed using any methods known in the art, either by solid phase or solution phase chemistry. Non-limiting examples for these methods are described hereby. Some of the preferred compounds of the present invention may conveniently be prepared using solution phase synthesis methods. Other methods known in the art to prepare compounds like those of the present invention, can be used and are comprised in the scope of the present invention.

The amino acid residues described herein are preferred to be in the “L” isomeric form. However, residues in the “D” isomeric form can be substituted for any L-amino acid residue, as long as the peptide retains the desired functional property.

The amino acids used in this invention are those which are available commercially or are available by routine synthetic methods. Certain residues may require special methods for incorporation into the peptide, and either sequential, divergent and convergent synthetic approaches to the peptide sequence are useful in this invention.

Conservative substitution of amino acids as known to those skilled in the art are within the scope of the present invention. Conservative amino acid substitutions includes replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

The pharmaceutical composition will be administered according to known modes of peptide administration, including oral, intravenous, subcutaneous, intraarticular, intramuscular, inhalation, intranasal, intrathecal, intradermal, transdermal or other known routes. The dosage administered will be dependent upon the age, sex, health condition and weight of the recipient, and the nature of the effect desired.

The peptides of the invention for use in therapy are typically formulated for administration to patients with a pharmaceutically acceptable carrier or diluent to produce a pharmaceutical composition. The formulation will depend upon the nature of the peptide and the route of administration but typically they can be formulated for topical, parenteral, intramuscular, intravenous, intraperitoneal, intranasal inhalation, lung inhalation, intradermal or intra-articular administration. The peptide may be used in an injectable form. It may therefore be mixed with any pharmaceutically acceptable vehicle which is suitable for an injectable formulation, preferably for a direct injection at the site to be treated, although it may be administered systemically.

The pharmaceutically acceptable carrier or diluent may be, for example, sterile isotonic saline solutions, or other isotonic solutions such as phosphate-buffered saline. The peptides of the present invention may be admixed with any suitable binder, lubricant, suspending agent, coating agent, solubilizing agent. It is also preferred to formulate the peptide in an orally active form.

Tablets or capsules of the peptides may be administered singly or two or more at a time, as appropriate. It is also possible to administer the peptides in sustained release formulations.

Typically, the physician will determine the actual dosage which will be most suitable for an individual patient and it will vary with the age, weight and response of the particular patient.

There can, of course, be individual instances where higher or lower dosage ranges are merited, and such are within the scope of this invention.

Alternatively, the peptides of the invention, can be administered by inhalation or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilizers and preservatives as may be required.

For some applications, preferably the compositions are administered orally in the form of tablets containing excipients such as starch or lactose, or in capsules or ovules either alone or in admixture with excipients, or in the form of elixirs, solutions or suspensions containing flavoring or coloring agents. For such oral administration, the peptide may preferably formed into microcapsules or nanoparticles together with biocompatible polymers such as poly-lactic acid and the like.

The compositions (as well as the peptides alone) can also be injected parenterally, for example intravenously, intramuscularly or subcutaneously. In this case, the compositions will comprise a suitable carrier or diluent. For parenteral administration, the compositions are best used in the form of a sterile aqueous solution, which may contain other substances, for example enough salts or monosaccharides to make the solution isotonic with blood.

For buccal or sublingual administration the compositions may be administered in the form of tablets or lozenges which can be formulated in a conventional manner.

A composition according to the invention can be formulated for parenteral administration by injection or continuous infusion. Compositions for injection can be provided in unit dose form and can take a form such as suspension, solution or emulsion in oil or aqueous carriers and can contain formulating agents, such as suspending, stabilizing and/or dispersing agents. Alternatively, the active constituent can be present in powder form for constitution with a suitable carrier, for example sterile pyrogen-free water, before use. The composition of the invention may be administrated directly into a body cavity adjacent to the location of the inflammatory area, such as the intraperitoneal cavity, or injected directly into or adjacent to the inflammatory area.

According to another aspect, the present invention provides a method of screening for active fragments of HSP60 capable of inducing Th2/3 T-cell responses. The method comprising: (a) applying a DNA construct encoding HSP70 to an animal in a sufficient amount to induce HSP70 expression in the animal; (b) obtaining T cells from said animal; (c) contacting the cells with a candidate HSP60 fragment for sufficient time for inducing cytokine secretion in said cells, and (d) determining the secretion of IL-10, TGFβ1 and IFNγ from said cells, wherein if the secretion of IL-10 and TGFβ1 is increased and the secretion of IFNγ is decreased than the candidate HSP60 fragment is capable of inducing Th2/3 T-cell responses.

It is to be noted that the compositions and methods of the present invention do not include the obligatory presence of the CpG motif disclosed in WO 02/16549, in DNA vaccines suitable for the treatment of ongoing autoimmune diseases.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Animals

Female Lewis rats were raised and maintained under pathogen-free conditions in the Animal Breeding Center of The Weizmann Institute of Science. One-month old rats were used for DNA vaccination experiments. The experiments were performed under the supervision and guidelines of the Animal Welfare Committee.

Antigens and Adjuvants

Peptides were synthesized as previously described (Quintana et al., 2002). The HSP65 peptide Mt176-190 used in these studies is EESNTFGLQLELTEG (SEQ ID NO:11), which contains the 180-188 epitope of HSP65 (van Eden et al., 1988). The panel of overlapping peptides spanning the whole HSP60 sequence has been described previously (Abulafia-Lapid et al., 1999). Purified recombinant HSP65 was generously provided by Prof. Ruurd van der Zee (Institute of Infectious Diseases and Immunology, Faculty of Veterinary Medicine, Utrecht, The Netherlands). Recombinant HSP60 was prepared in our laboratory as described (Quintana et al., 2000). Human recombinant HSP70 was purchased from Sigma (Rehovot, Israel). Recombinant glutathion-S-transferase (GST) and GST-HSP90 were prepared in our laboratory as described (Nemoto et al., 1997). M. tuberculosis Strain H37Ra and incomplete Freund's adjuvant (IFA) were purchased from Difco (Detroit, Mich., USA). Tuberculin purified protein derivative (PPD) and mycobacterial 71 kDa heat shock protein (HSP71) were provided by the Statens Seruminstitut (Copenhagen, Denmark). Ovalbumin (OVA) and Concanavalin A (Con A) were purchased from Sigma (Rehovot, Israel).

DNA Plasmids

The vector containing the human hsp60 gene (pHSP60) has been described (Quintana et al., 2000). The construct encoding Mycobacterium leprae HSP65 (pHSP65) was kindly provided by Dr. Douglas Lowrie (Medical Research Council, London, UK). Both vectors have been shown to be effective in inhibiting AA (Quintana et al., 2002; Ragno et al., 1997).

The full length cDNAs of the human hsp70 (accession number M11717, SEQ ID NO:12) and hsp90a (accession number NM_{—}005348, SEQ ID NO:13) genes were cloned into the pcDNA3 vector (Invitrogen, NV, Leek, The Netherlands). In brief, human hsp70 cDNA in pHLTR-HSP70 and hsp90 cDNA in pGEX-HSP90 were amplified by PCR using specific oligonucleotides containing restriction sites for the enzymes BamHI or XbaI. The amplicons and the pcDNA3 vector were purified and digested with BamHI and XbaI. The digested PCR products coding for HSP70 or HSP90 and the linearized pcDNA3 vector were ligated using a Rapid DNA Ligation Kit (Roche Diagnostics GmbH, Mannheim, Germany), according to the standard protocol given by the manufacturer. The ligated plasmids were transformed into Escherichia coli, and later, sequenced to confirm correct insertion of the cDNA. The pHSP70 and pHSP90 plasmids were checked in the TNT® Quick Coupled Transcription/Translation System (Promega, Madison, USA) according to the manufacturer's instructions using [³⁵S]-methionine (Amersham, Buckinghamshire, UK). The ³⁵S-labeled transcription products were analyzed by SDS-PAGE and autoradiography.

Plasmid DNA was prepared in large scale and injected after pretreatment with cardiotoxin (Sigma, Rehovot, Israel) as previously described (Quintana et al., 2002). Briefly, rats were vaccinated in the quadriceps three times (on days −40, −26-12 relative to AA induction) with 150 μg of pcDNA3, pHSP60, pHSP65, pHSP70 or pHSP90. Endotoxin levels were checked by Limulus Amoebocyte Lysate and found always to be under acceptable levels for in vivo use (less than 0.02 EU/μg DNA). AA was induced 12 days after the last injection of DNA. The empty vector pcDNA3 was used as a DNA vaccination control.

Detection of HSP70- and HSP90-Specific Antibodies

Blood samples were collected at the beginning and 12 days after the end of the regime of DNA vaccination, and 12 days after the induction of AA. Serum was prepared as previously described (Quintana et al., 2002) and kept at −20° C. until used. HSP70- or HSP90-specific antibodies were measured using an ELISA assay in flat-bottom microtiter plates (Maxisorb, Nunc, Denmark) coated overnight with 500 ng/well of HSP70 or OVA, or 1 μg/well of GST-HSP90 or GST in carbonate buffer at 4° C. Non-specific binding was blocked by incubation with 1% skim milk for 2 hr at 37° C. and serum samples were added diluted 1/100 and incubated 3 hr at 37° C. Bound IgG antibodies were detected using alkaline phosphatase-conjugated goat anti-rat IgG (Jackson ImmunoResearch, USA) together with Sigma's substrate for alkaline phosphatase.

AA Induction and Assessment

AA was induced as described (Yang et al., 1992), by immunizing Lewis rats with 1 mg per rat of heat-killed Mt strain H37Ra (Difco). Each experimental and control group contained at least 8 rats. The day of AA induction was designated as day 0, and disease severity was assessed by direct observation of all 4 limbs in each animal. A relative score between 0 and 4 was assigned to each limb, based on the degree of joint inflammation, redness and deformity; thus the maximum possible score for an individual animal was 16 (Quintana et al., 2002). The mean AA score (±SEM) is shown for each experimental group. The person who scored the disease was blind to the identity of the groups. Arthritis was also quantified by measuring hind limb diameter with a caliper. Measurements were taken on the day of the induction of AA and 26 days later (at the peak of AA); the results are presented as the mean ±SEM of the difference between the two values for all the animals in each group. The experiments were repeated at least 3 times and produced similar results.

T-Cell Proliferation

T-cell proliferation assays were performed at day 26 after the induction of AA, when the disease is at its peak, as previously described (Quintana et al., 2002). Briefly, popliteal and inguinal lymph node cells (LNC), were cultured in quadruplicates in 200 μl round bottom microtiter wells (Costar Corp., Cambridge, USA) at 2×10⁵ cells per well with or without antigen. The T-cell mitogen Concanavalin A (Con A) was used as a positive control for T-cell proliferation. Cultures were incubated for 96 hr at 37° C. in a humidified atmosphere of 5% CO₂. T-cell responses were detected by the incorporation of [methyl-3H]-thymidine (Amersham, Buckinghamshire, UK; 1 μCi/well), which was added to the wells for the last 18 hr. The stimulation index (SI) was computed as the ratio of the mean c.p.m. of antigen- or mitogen-containing wells to control wells cultured with medium alone. The results of T-cell proliferation experiments are shown as SI±SEM, T-cell responses with SI<2 were considered not significant.

Cytokine Assays

Supernatants were collected after 72 hrs of stimulation with each of the antigens tested. Rat IL-10 and IFNγ were quantitated in culture supernatants by enzyme-linked immunosorbent assay (ELISA) using Pharmingen's OPTEIA kit (Pharmingen, San Diego, USA) as described (Yang et al., 1992). Rat TGFβ1 was quantified using the TGFβ1 Eₘₐₓ® ImmunoAssay System (Promega, Madison, USA) according to the manufacturer's instructions. Cytokine levels are expressed as pg/ml based on calibration curves constructed using recombinant cytokines as standards. The lower limits of detection for the experiments described in this paper were 15 pg/ml for TGFβ1, IL-10 and IFNγ.

Statistical Significance

The InStat 2.01 program was used for statistical analysis. Student's t-test and the Mann-Whitney test were carried out to assay significant differences between the different experimental groups.

### Example 1

### pHSP70 and pHSP90 are Expressible and Immunogenic

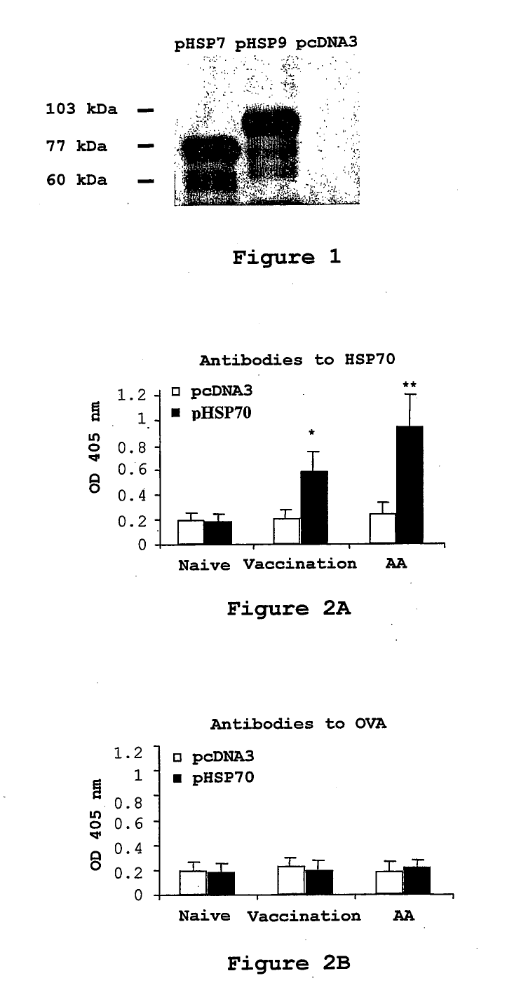

The pHSP70 and pHSP90 constructs were transcribed/translated in vitro in the presence of [³⁵S]-methionine, and the products of translation were analysed by SDS-PAGE followed by autoradiography. No ³⁵S-labeled protein was detected in the transcription/translation products induced by the control pcDNA3 vector, but products of 70 and 90 kDa were present in the samples induced by pHSP70 and pHSP90, respectively (FIG. 1). A few minor bands were also detected, both for the HSP70 and the HSP90 preparations, but they were likely to be degradation products because they were recognized by specific antibodies directed against HSP70 or HSP90.

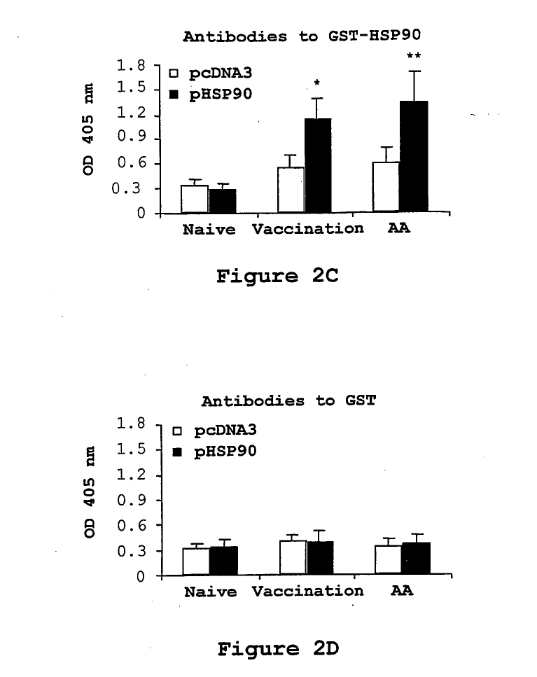

It was also tested whether vaccination with the pHSP70 or pHSP90 constructs could induce antigen-specific antibodies. Eight rats were vaccinated three times (5, 19 and 33 days after the pre-treatment with cardiotoxin) with pHSP70, pHSP90 or with the empty vector pcDNA3. Serum samples were collected at the day of the first DNA vaccination, and 12 days later, and IgG antibodies to HSP70, OVA, GST-HSP90 or GST were quantified by ELISA. DNA vaccination with pHSP70 or pHSP90 induced significant levels of IgG antibodies specific for HSP70 (FIG. 2A) or GST-HSP90 (FIG. 2C). None of the experimental groups had significant levels of IgG antibodies to OVA (FIG. 2B) or to GST (FIG. 2D).

To test the effect of AA induction on these antibodies, the rats were immunized with Mt, and the serum IgG antibodies were measured 12 days later. The levels of the antibodies induced by DNA-vaccination were further enhanced by the induction of AA (FIGS. 2A and 2C). These results demonstrate that the pHSP70 and pHSP90 constructs are functional in vitro and can induce antigen-specific immune responses in vivo. Moreover, AA induction seems to boost the antibodies.

### Example 2

### DNA Vaccination with HSP70 or HSP90 Inhibits AA

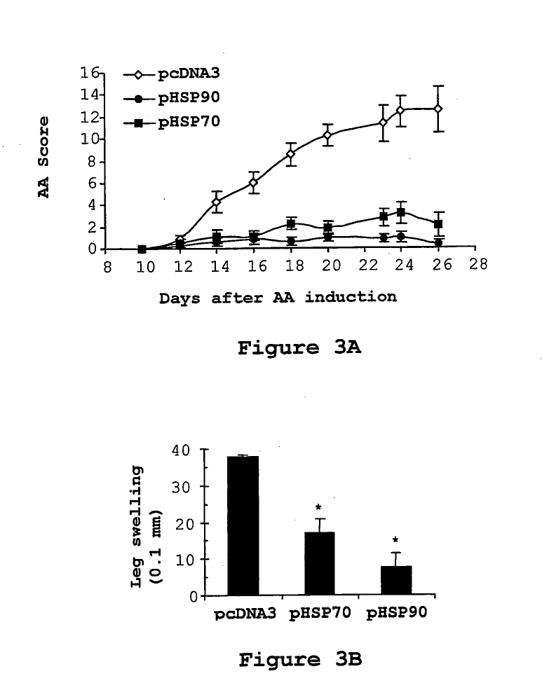

The effects on AA of DNA vaccination with pHSP70 or pHSP90 was investigated. The empty control vector pcDNA3 had no effect on the development of AA (FIG. 3A), as previously reported (Quintana et al., 2002). But, pHSP70 or pHSP90 vaccination induced a significantly milder arthritis, in onset of disease, clinical score (FIG. 3A) and ankle swelling (FIG. 3B). The mean maximum score was 14.7±0.9 in the pcDNA3-treated rats, compared to 4.5±1.1 in the pHSP70-treated rats and 4.5±1.2 in the pHSP90-treated rats (p<0.001 for both test groups compared to the pcDNA3 group). The mean day of onset was 12.1±0.1 in the pcDNA3-treated rats, compared to 16.3±1.5 in pHSP70-treated rats and 16.2±1.8 in pHSP90-treated rats (p<0.001 for both test groups compared to the pcDNA3 group). Thus, DNA vaccination with vectors encoding mammalian HSP70 or HSP90 can significantly inhibit AA.

### Example 3

### Arthritogenic Immune Response in Vaccinated Rats: T-Cell Proliferation to Mt Antigens

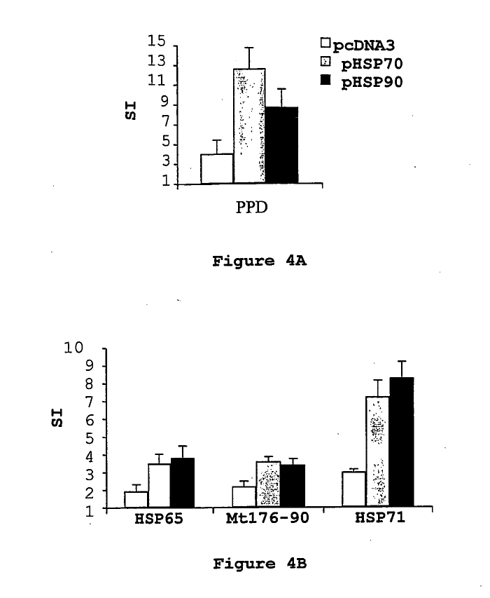

The inhibition of AA by DNA vaccination (Quintana et al., 2002; Quintana et al., 2003) or other means (Yang et al., 1992) has been associated with increased proliferation to Mt-derived antigens. The LNC proliferative responses were studied, 26 days after the induction of AA, of rats vaccinated with control pcDNA3, pHSP70 or pHSP90, to a panel of relevant mycobacterial and mammalian antigens. FIG. 4 shows that the LNC of the rats protected by pHSP70 or pHSP90 vaccination (FIG. 3) showed stronger proliferative responses than did the control rats to PPD, mycobacterial HSP71, HSP65 and peptide Mt176-90—antigens known to be targeted or associated with AA (van Eden et al., 1988, Holoshitz et al., 1983). None of the experimental groups showed significant T-cell responses to OVA, and they did not differ in their responses to Con A. Thus, inhibition of AA by vaccination with pHSP70 or pHSP90, have been found by the present inventors following pHSP60 vaccination (Quintana et al., 2002), is associated with increased T-cell proliferation against a variety of mycobacterial antigens associated with AA.

### Example 4

### Arthritogenic Immune Response in Vaccinated Rats: Cytokine Secretion to Mt Antigens

The induction of AA has been reported to up-regulate antigen-specific IFNγ secretion, while immunomodulation of AA has been associated with the down-regulation of IFNγ secretion and the up-regulation of Th2/3-like cytokines in response to relevant Mt antigens (Quintana et al., 2002). The effects of DNA vaccination with pHSP70 or pHSP90 on the profile of cytokine secretion 26 days after the induction of AA were studied.

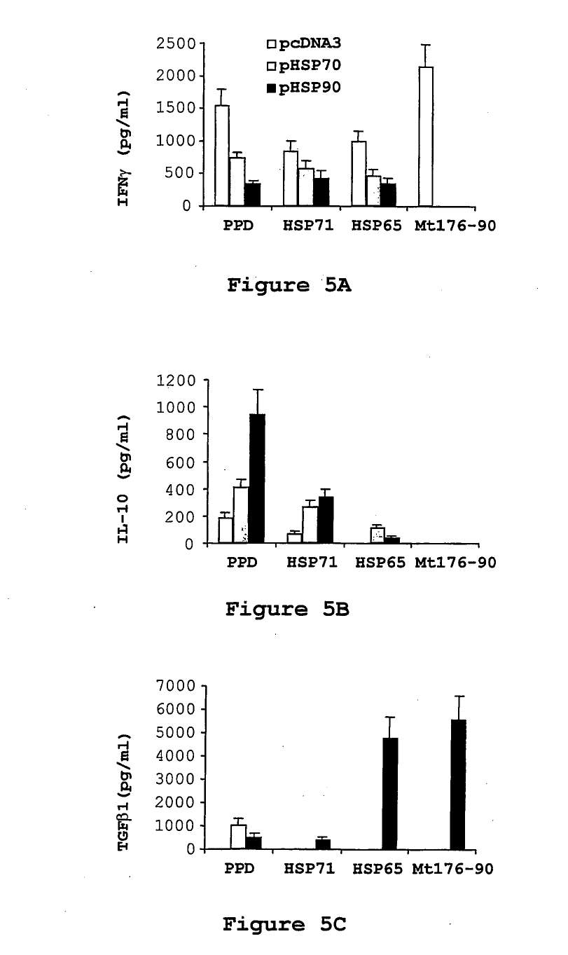

LNC from pHSP70 and pHSP90-vaccinated rats secreted significantly lower amounts of IFNγ upon stimulation with mycobacterial PPD, HSP71, HSP65 or its T-cell epitope Mt176-90 than did control pcDNA3-treated rats, with unmodified AA (FIG. 5A). In contrast, LNC from pHSP70- and pHSP90-vaccinated rats secreted IL-10 in response to stimulation with PPD, HSP65 or HSP71 (FIG. 5B). Stimulation with PPD, HSP71, HSP65 or MT176-90 led to the secretion of significant amounts of TGFβ1 from LNC of pHSP90-vaccinated rats, in particular against HSP65 and its peptide Mt176-90 (FIG. 5C). LNC of pHSP70-vaccinated rats only secreted TGFβ1 upon activation with PPD (FIG. 5C).

In summary, inhibition of AA by pHSP70 or pHSP90 vaccination was associated with a decrease in the secretion of IFNγ and an increase in IL-10 and/or TGFβ1 secretion in the Mt-specific T-cell response.

### Example 5

### Mammalian HSP-Specific Immune Responses

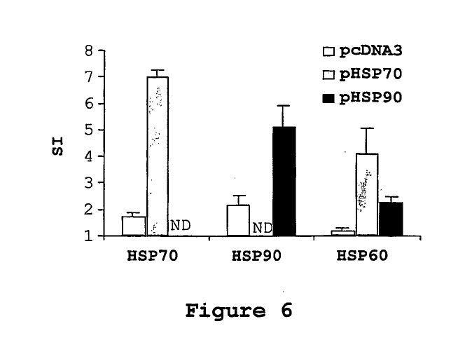

It has previously been reported that the inhibition of AA by HSP60 DNA vaccination involves the activation of T-cells reactive to HSP60 (Quintana et al., 2002; Quintana et al., 2003). The effect of pHSP70 or pHSP90 vaccination on T-cell responses to HSP70, HSP90 and HSP60 was studied. Both DNA vaccines, pHSP70 and pHSP90, induced antigen-specific proliferative responses: pHSP70-vaccinated rats manifested T-cell responses to HSP70 (FIG. 6) and not to OVA, and pHSP90-vaccinated rats manifested T-cell responses to GST-HSP90 (FIG. 6), and not the control protein GST. Unexpectedly, DNA-vaccination with pHSP70 or pHSP90 induced modest but significant T-cell proliferative responses to HSP60 (FIG. 6).

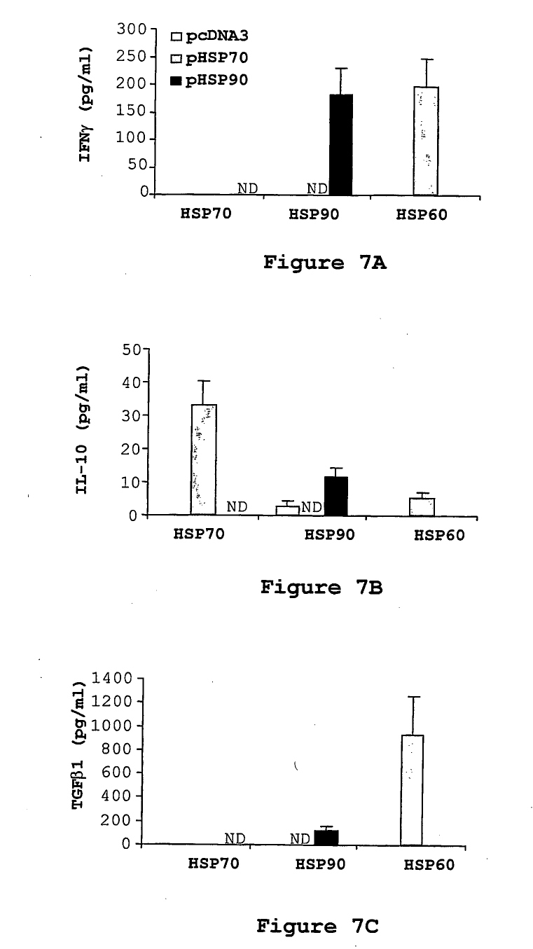

The effect of pHSP70 and pHSP90 vaccination on the cytokine responses to HSP70, HSP90 and HSP60 was studied. FIG. 7 shows that vaccination with pHSP90 induced HSP90-specific cells that secreted. IFNγ, IL-10 and TGFβ1 following stimulation with HSP90. LNC from pHSP70 rats secreted IL10 upon stimulation with HSP70, but not IFNγ or TGFβ1 (FIG. 7). Strikingly, LNC taken from pHSP70-vaccinated rats secreted IFNγ, TGFβ1 and a lesser amount of IL-10 upon activation with HSP60 (FIG. 7). Thus, vaccination with pHSP90 or pHSP70 induced T-cell responses against the HSP encoded by the DNA, but also activated HSP60-specific T-cell immunity. The cross-modulation with HSP60 was apparently stronger following vaccination with pHSP70.

### Example 6

### pHSP70- and pHSP60-Vaccinated Rats Recognize Different HSP60 T-Cell Epitopes

It was previously reported that the suppression of AA by pHSP60 DNA vaccination was associated with T-cell reactivity to a single HSP60 peptide epitope, Hu3 (Quintana et al., 2003). Since pHSP70-vaccination induced strong T-cell responses to HSP60 (FIGS. 6 and 7), the proliferation of LNC to a panel of overlapping peptides spanning the human HSP60 sequence (Abulafia-Lapid et al., 1999) was studied. Control LNC were prepared from rats vaccinated with pcDNA3 (negative control) or pHSP60 (positive control). The rats were challenged with Mt to induce AA, and the responses were assayed on day 26. Table I shows that the LNC taken from pHSP60-vaccinated rats responded only to peptide Hu3 (aa 31-50); LNC from pHSP70 or pcDNA-vaccinated rats did not respond to Hu3; the LNC from pHSP70-vaccinated rats responded to several other HSP60 peptides: Hu19 (aa 271-290), Hu24 (aa 346-365), Hu25 (aa 361-380), Hu27 (aa 391-410), Hu28 (aa 406-425), Hu30 (aa 436-455), Hu32 (aa 466-485), Hu33 (aa 481-500) and Hu34 (aa 271-290). In summary, these results show that LNC from rats vaccinated with pHSP70 recognized different HSP60 T-cell epitopes than do LNC from rats vaccinated with HSP60 itself. Vaccination with pHSP70 does activate T-cells reactive to HSP60, but the T-cells respond to peptides other than peptide Hu3, which is the epitope characteristic of pHSP60 vaccination.

TABLE I

LNC proliferative response to
overlapping peptides of HSP60.

| | | | |
|---|---|---|---|
| SEQ ID | pcDNA3 | pHSP60 | pHSP70 |

| | | | |
|---|---|---|---|
| Antigen | NO: | Sequence | Stimulation Index (SI) |

| | | | | | |
|---|---|---|---|---|---|
| Hu3 (31-50) | 10 | KFGADARALMLQGVDLLADA | — | 4.5 ± 0.8 | — |
| Hu19 (271-290) | 1 | LVIIAEDVDGEALSTLVLNR | — | — | 3.6 ± 0.3 |
| Hu24 (346-365) | 2 | GEVIVTKDDAMLLKGKGDKA | — | — | 4.3 ± 0.6 |
| Hu25 (361-380) | 3 | KGDKAQIEKRIQEIIEQLDV | — | — | 3.5 ± 0.4 |
| Hu27 (391-410) | 4 | NERLAKLSDGVAVLKVGGTS | — | — | 3.2 ± 0.4 |
| Hu28 (406-425) | 5 | VGGTSDVEVNEKKDRVTDAL | — | — | 3.2 ± 0.3 |
| Hu30 (436-455) | 6 | IVLGGGCALLRCIPALDSLT | — | — | 3.1 ± 0.5 |
| Hu32 (466-485) | 7 | EIIKRTLKIPANTIAKNAGV | — | — | 3.4 ± 0.6 |
| Hu33 (481-500) | 8 | KNAGVEGSLIVEKIMQSSSE | — | — | 4.1 ± 0.4 |
| Hu34 (496-515) | 9 | QSSSEVGYDAMAGDFVNMVE | — | — | 3.4 ± 0.3 |

### Example 7

### DNA Vaccination with HSP60 or HSP65 Boosts Immune Responses to Both Mammalian and Mycobacterial HSP70

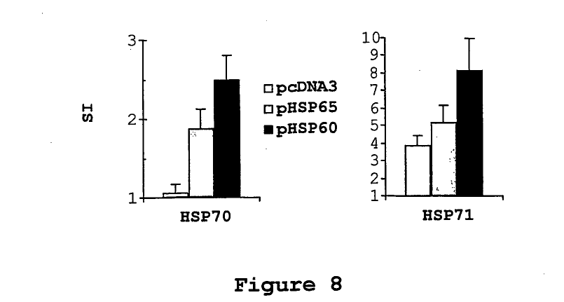

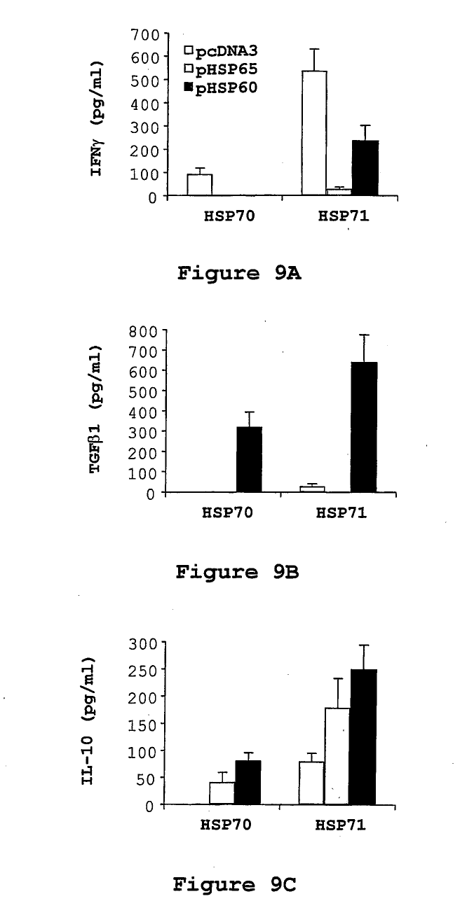

To further investigate immune cross-talk between HSP60 and HSP70, T-cell reactivity to mammalian HSP70 and mycobacterial HSP71 26 days after the induction of AA in rats that had been vaccinated with pHSP60, pHSP65 or pcDNA3 was studied. LNC taken from pHSP60- or pHSP65-vaccinated rats manifested increased proliferative responses to HSP70 and HSP71 (FIG. 8); the rats did not show significant T-cell responses to OVA, and the groups did not differ in their responses to Con A. Moreover, LNC taken from pHSP60- or pHSP65-vaccinated rats secreted significantly less IFNγ upon stimulation with HSP71 (FIG. 9A), while they secreted detectable amounts of IL-10 upon stimulation with HSP70 (FIG. 9B), and significantly higher levels of IL-10 upon activation with HSP71, compared with LNC taken from pcDNA3-vaccinated rats (FIG. 9B). Only LNC taken from pHSP60-vaccinated rats secreted significant amounts of TGFβ1 upon stimulation with HSP70 or HSP71 (FIG. 9C). Thus, DNA vaccination with pHSP60 or pHSP65 activates Th2/3 T-cell responses to HSP71 and to HSP70. The cross talk between HSP60 and HSP70 is mutual.

### Example 8

### DNA Vaccination with pHSP70 Triggered the Release of Endogenous pHSP60 to the Circulation

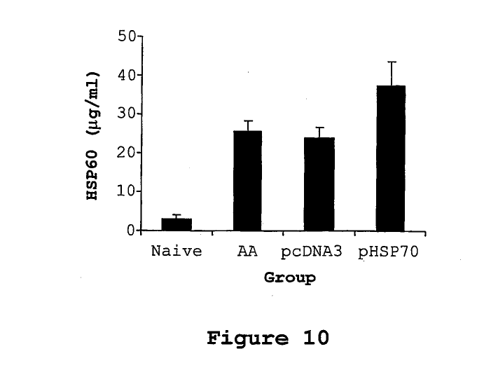

Serum HSP60 has been linked to inflammation, therefore HSP60 levels in serum after the induction of AA in rats vaccinated with HSP70 or pcDNA3 were assayed. Blood samples were collected at day 26 after the induction of AA, and serum HSP60 was quantified in serum as reported (Quintana et al., 2002). FIG. 10 shows that AA itself increased serum HSP60, and the HSP60 levels were further increased by vaccination with pHSP70. Thus, self-vaccination with endogenous HSP60 triggered by the pHSP70 and pHSP90 DNA vaccines might play a role in the inhibitory effect of HSP70 DNA vaccination on AA.

### Example 9

### DNA Vaccination with Recombinant Constructs Encoding HSP60 Fragments

A recombinant construct is prepared, comprising the nucleic acid sequence: ATTGTTTTGGGAGGGGGTTGTGCCCTCCTTCGATGCATTCCAGCCTTGGACTC ATTGACT (nucleic acids 1351-1410 of SEQ ID NO:15) encoding amino acids 436-455 of human HSP60 (SEQ ID NO:6), incorporated into pcDNA3. Lewis rats are vaccinated with the resulting recombinant construct or with the control vector pcDNA3, AA is induced, and arthritis scores are assessed every two or three days starting at day 10.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art.

Accordingly, it is intended to embrace all such alternatives, modifications and variations that fall within the spirit and broad scope of the appended claims.

### REFERENCES

1. van Eden, W., J. E. Thole, R. van der Zee, A. Noordzij, J. D. van Embden, E. J. Hensen, and I. R. Cohen. Nature 331:171, 1988.
2. van Eden, W., J. Holoshitz, Z. Nevo, A. Frenkel, A. Klajman, and I. R. Cohen. Proc Natl Acad Sci USA 82:5117, 1985.
3. Holoshitz, J., A. Matitiau, and I. R. Cohen. J Clin Invest 73:211, 1984.
4. Holoshitz, J., Y. Naparstek, A. Ben-Nun, and I. R. Cohen. Science 219:56, 1983.
5. Billingham, M. E., S. Carney, R. Butler, and M. J. Colston. J Exp Med 171:339, 1990.
6. Hogervorst, E. J., L. Schouls, J. P. Wagenaar, C. J. Boog, W. J. Spaan, J. D. van Embden, and W. van Eden. Infect Immun 59:2029, 1991.
7. Ragno, S., M. J. Colston, D. B. Lowrie, V. R. Winrow, D. R. Blake, and R. Tascon. Arthritis Rheum 40:277, 1997.
8. Moudgil, K. D., T. T. Chang, H. Eradat, A. M. Chen, R. S. Gupta, E. Brahn, and E. E. Sercarz. J Exp Med 185:1307, 1997.
9. Anderton, S. M., R. van der Zee, B. Prakken, A. Noordzij, and W. van Eden. J Exp Med 181:943, 1995.
10. Yang, X. D., J. Gasser, and U. Feige. Clin Exp Immunol 87:99, 1992.
11. van Eden, W., U. Wendling, L. Paul, B. Prakken, P. van Kooten, and R. van der Zee. Cell Stress Chaperones 5:452, 2000.
12. Lopez-Guerrero, J. A., J. P. Lopez-Bote, M. A. Ortiz, R. S. Gupta, E. Paez, and C. Bernabeu. Infect Immun 61:4225, 1993.
13. Lopez-Guerrero, J. A., M. A. Ortiz, E. Paez, C. Bernabeu, and J. P. Lopez-Bote. Arthritis Rheum 37:1462, 1994.
14. Quintana, F. J., P. Carmi, F. Mor, and I. R. Cohen. J Immunol 169:3422, 2002.
15. Quintana F J, Carmi P, Mor F, Cohen I R. J. Immunol. 2003 Oct. 1; 171(7):3533-41, 2003.
16. Abulafia-Lapid, R., D. Elias, I. Raz, Y. Keren-Zur, H. Atlan, and I. R. Cohen. J Autoimmun 12:121, 1999.
17. Quintana, F. J., A. Rotem, P. Carmi, and I. R. Cohen. J Immunol 165:6148, 2000.
18. Nemoto, T., N. Sato, H. Iwanari, H. Yamashita, and T. Takagi. J Biol Chem 272:26179, 1997.
19. Sambrook et al., Molecular Cloning: A Laboratory Manual, Cold Spring Harbor Labs Press, 1989.
20. Wolff et al. Science 247, 1465-1468, 1990,
21. Stribling et al. Proc. Natl. Acad. Sci. USA 189:11277-11281, 1992.

1-90. (canceled)

91. A DNA vaccine for treating a T cell-mediated inflammatory autoimmune disease comprising a recombinant construct comprising a nucleic acid sequence encoding a fragment of heat shock protein 60 (HSP60) characterized in that it reacts with T cells isolated from an animal vaccinated with HSP70 to induce Th2/3 T-cell responses.

92. The DNA vaccine of claim 91, wherein the fragment is derived from human HSP60.

93. The DNA vaccine of claim 92, wherein the fragment comprises amino acid sequence selected from: amino acids 271-290 of human HSP60 (SEQ ID NO:1), amino acids 346-365 of human HSP60 (SEQ ID NO:2), amino acids 361-380 of human HSP60 (SEQ ID NO:3), amino acids 391-410 of human HSP60 (SEQ ID NO:4), amino acids 406-425 of human HSP60 (SEQ ID NO:5), amino acids 436-455 of human HSP60 (SEQ ID NO:6), amino acids 466-485 of human HSP60 (SEQ ID NO:7), amino acids 481-500 of human HSP60 (SEQ ID NO:8) and amino acids human 496-515 of HSP60 (SEQ ID NO:9).

94. The DNA vaccine of claim 91, wherein the nucleic acid sequence is operatively linked to one or more transcription control sequences in a suitable expression system enabling in vivo expression of the encoded fragment in a human host.

95. The DNA vaccine of claim 94, wherein the transcription control sequences are selected from the group consisting of: RSV control sequences, CMV control sequences, retroviral LTR sequences, SV-40 control sequences and β-actin control sequences.

96. The DNA vaccine of claim 91, wherein the recombinant construct is incorporated into an eukaryotic expression vector.

97. A recombinant construct comprising a nucleic acid sequence encoding a fragment of HSP60 characterized in that it reacts with T cells isolated from an animal vaccinated with HSP70 to induce Th2/3 T-cell responses, the nucleic acid sequence being operatively linked to one or more transcription control sequences.

98. The construct of claim 97, wherein the fragment comprises amino acid sequence selected from: amino acids 271-290 of human HSP60 (SEQ ID NO:1), amino acids 346-365 of human HSP60 (SEQ ID NO:2), amino acids 361-380 of human HSP60 (SEQ ID NO:3), amino acids 391-410 of human HSP60 (SEQ ID NO:4), amino acids 406-425 of human HSP60 (SEQ ID NO:5), amino acids 436-455 of human HSP60 (SEQ ID NO:6), amino acids 466-485 of human HSP60 (SEQ ID NO:7), amino acids 481-500 of human HSP60 (SEQ ID NO:8) and amino acids 496-515 of human HSP60 (SEQ ID NO:9).

99. The construct of claim 97, wherein the recombinant construct is incorporated into a eukaryotic expression vector.

100. The construct of claim 101, wherein the eukaryotic expression vector is selected from the group consisting of: pcDNA3, pcDNA3.1(+/−), pZeoSV2(+/−), pSecTag2, pDisplay, pEF/myc/cyto, pCMV/myc/cyto, pCR3.1, pCI, PBK-RSV, PBK-CMV and pTRES.

101. A pharmaceutical composition comprising (a) a recombinant construct according to claim 97; and (b) a pharmaceutically acceptable carrier.

102. The composition of claim 101, wherein the carrier comprises a delivery vehicle that delivers the nucleic acid sequences to a subject, wherein said delivery vehicle is selected from the group consisting of liposomes, micelles, emulsions and cells.

103. The composition of claim 101, wherein the nucleic acid sequence is operatively linked to one or more transcription control sequences.

104. The composition of claim 101, wherein the recombinant construct is incorporated into a eukaryotic expression vector.

105. The composition of claim 101, wherein the fragment comprises amino acid sequence selected from: amino acids 271-290 of human HSP60 (SEQ ID NO:1), amino acids 346-365 of human HSP60 (SEQ ID NO:2), amino acids 361-380 of human HSP60 (SEQ ID NO:3), amino acids 391-410 of human HSP60 (SEQ ID NO:4), amino acids 406-425 of human HSP60 (SEQ ID NO:5), amino acids 436-455 of human HSP60 (SEQ ID NO:6), amino acids 466-485 of human HSP60 (SEQ ID NO:7), amino acids 481-500 of human HSP60 (SEQ ID NO:8) and amino acids 496-515 of human HSP60 (SEQ ID NO:9).

106. A pharmaceutical composition comprising (a) a peptide fragment of HSP60 characterized in that it reacts with T cells isolated from an animal vaccinated with HSP70 to induce Th2/3 T-cell responses; and (b) a pharmaceutically acceptable carrier.

107. The composition of claim 106, wherein the fragment is derived from human HSP60.

108. The composition of claim 106, wherein the fragment comprises amino acid sequence selected from: amino acids 361-380 of human HSP60 (SEQ ID NO:3), amino acids 391-410 of human HSP60 (SEQ ID NO:4), amino acids 406-425 of human HSP60 (SEQ ID NO:5), and amino acids 496-515 of human HSP60 (SEQ ID NO:9).

109. A method of treating or preventing the symptoms of a T cell-mediated inflammatory autoimmune disease, comprising administering to a subject in need thereof a therapeutically effective amount of a pharmaceutical composition comprising a recombinant construct according to claim 97.

110. The method of claim 109, wherein the nucleic acid sequence is operatively linked to one or more transcription control sequences.

111. The method of claim 109, wherein the fragment comprises amino acid sequence selected from: amino acids 271-290 of human HSP60 (SEQ ID NO:1), amino acids 346-365 of human HSP60 (SEQ ID NO:2), amino acids 361-380 of human HSP60 (SEQ ID NO:3), amino acids 391-410 of human HSP60 (SEQ ID NO:4), amino acids 406-425 of human HSP60 (SEQ ID NO:5), amino acids 436-455 of human HSP60 (SEQ ID NO:6), amino acids 466-485 of human HSP60 (SEQ ID NO:7), amino acids 481-500 of human HSP60 (SEQ ID NO:8) and amino acids 496-515 of human HSP60 (SEQ ID NO:9).

112. The method of claim 109, wherein the T cell-mediated inflammatory autoimmune disease is selected from the group consisting of: rheumatoid arthritis, collagen II arthritis, multiple sclerosis, autoimmune neuritis, systemic lupus erythematosus, psoriasis, juvenile onset diabetes, Sjogren's disease, thyroid disease, sarcoidosis, autoimmune uveitis, inflammatory bowel disease (Crohn's and ulcerative colitis) and autoimmune hepatitis.

113. The method of claim 109 wherein the disease is arthritis.

114. The method of claim 111 wherein the disease is arthritis.

115. The method of claim 109, wherein the subject is selected from the group consisting of humans and non-human mammals.

116. The method of claim 109, wherein the pharmaceutical composition is administered to said subject in a manner selected from the group consisting of:
a) administering said composition at the time of appearance of disease symptoms; and
b) administering said composition prior to the appearance of disease symptoms.

117. The method of claim 109, wherein the pharmaceutical composition is administered in a manner selected from the group consisting of: intravenous injection, intramuscular injection, aerosal, oral, percutaneous or topical administration.

118. A method for treating or preventing the symptoms of a T cell-mediated inflammatory autoimmune disease comprising the steps of (a) obtaining cells from a subject; (b) transfecting the cells in vitro with a recombinant construct according to claim 97; and (c) reintroducing the transfected cells to the subject, thereby treating the disease.

119. The method of claim 118, wherein the fragment comprises amino acid sequence selected from: amino acids 271-290 of human HSP60 (SEQ ID NO:1), amino acids 346-365 of human HSP60 (SEQ ID NO:2), amino acids 361-380 of human HSP60 (SEQ ID NO:3), amino acids 391-410 of human HSP60 (SEQ ID NO:4), amino acids 406-425 of human HSP60 (SEQ ID NO:5), amino acids 436-455 of human HSP60 (SEQ ID NO:6), amino acids 466-485 of human HSP60 (SEQ ID NO:7), amino acids 481-500 of human HSP60 (SEQ ID NO:8) and amino acids 496-515 of human HSP60 (SEQ ID NO:9).

120. A method for treating or preventing a T cell-mediated inflammatory autoimmune disease comprising the steps of (a) obtaining cells from a subject; (b) infecting the cells in vitro with a virus comprising a recombinant construct according to claim 97; and (c) reintroducing the infected cells to the subject, thereby treating the disease.

121. The method of claim 120, wherein the fragment comprises amino acid sequence selected from: amino acids 271-290 of human HSP60 (SEQ ID NO:1), amino acids 346-365 of human HSP60 (SEQ ID NO:2), amino acids 361-380 of human HSP60 (SEQ ID NO:3), amino acids 391-410 of human HSP60 (SEQ ID NO:4), amino acids 406-425 of human HSP60 (SEQ ID NO:5), amino acids 436-455 of human HSP60 (SEQ ID NO:6), amino acids 466-485 of human HSP60 (SEQ ID NO:7), amino acids 481-500 of human HSP60 (SEQ ID NO:8) and amino acids 496-515 of human HSP60 (SEQ ID NO:9).

122. A method of treating or preventing a T cell-mediated inflammatory autoimmune disease, comprising administering to a subject in need thereof a therapeutically effective amount of a pharmaceutical composition according to claim 16.

123. The method of claim 122, wherein the fragment comprises amino acid sequence selected from: amino acids 271-290 of human HSP60 (SEQ ID NO:1), amino acids 346-365 of human HSP60 (SEQ ID NO:2), amino acids 361-380 of human HSP60 (SEQ ID NO:3), amino acids 391-410 of human HSP60 (SEQ ID NO:4), amino acids 406-425 of human HSP60 (SEQ ID NO:5), amino acids 436-455 of human HSP60 (SEQ ID NO:6), amino acids 466-485 of human HSP60 (SEQ ID NO:7), amino acids 481-500 of human HSP60 (SEQ ID NO:8) and amino acids 496-515 of human HSP60 (SEQ ID NO:9).

124. The method of claim 122, wherein the T cell-mediated inflammatory autoimmune disease is selected from the group consisting of: rheumatoid arthritis, collagen II arthritis, multiple sclerosis, autoimmune neuritis, systemic lupus erythematosus, psoriasis, juvenile onset diabetes, Sjogren's disease, thyroid disease, sarcoidosis, autoimmune uveitis, inflammatory bowel disease (Crohn's and ulcerative colitis) and autoimmune hepatitis.

125. The method of claim 122, wherein the pharmaceutical composition is administered to said subject in a manner selected from the group consisting of:
a) administering said composition at the time of appearance of disease symptoms; and
b) administering said composition prior to the appearance of disease symptoms.

126. The method of claim 122, wherein the pharmaceutical composition is administered by intravenous injection, intramuscular injection, aerosal, oral, percutaneous or topical administration.

127. The method of claim 122 wherein the disease is arthritis.

128. A method of screening for active fragments of HSP60 capable of inducing Th2/3 T-cell responses comprising:
(a) applying a DNA construct encoding HSP70 to an animal in a sufficient amount to induce HSP70 expression in the animal;
(b) obtaining T cells from said animal;
(c) contacting the cells with a candidate HSP60 fragment for sufficient time for inducing cytokine secretion in said cells; and
(d) determining the secretion of IL-10, TGFβ1 and IFNγ from said cells, wherein if the secretion of IL-10 and TGFβ1 is increased and the secretion of IFNγ is decreased then the candidate HSP60 fragment is capable of inducing Th2/3 T-cell responses.
